(19) United States
(12) Patent Application Publication
(10) Pub. No.: US 2017/0181956 A1
(43) Pub. Date: Jun. 29, 2017

(54) HAIR DENSIFYING AGENT AND DISPENSER

(71) Applicant: Mana Products, Inc., Long Island City, NY (US)

(72) Inventors: George Lambridis, Wayne, NJ (US); Julio Pina, Woodside, NY (US); Ann Kohatsu, Long Island City, NY (US); Victoria Colangelo, Long Island City, NY (US); Nikos Mouyiaris, Long Island City, NY (US)

(21) Appl. No.: 15/444,805

(22) Filed: Feb. 28, 2017

Related U.S. Application Data
(63) Continuation of application No. PCT/US16/68877, filed on Dec. 28, 2016.
(60) Provisional application No. 62/272,552, filed on Dec. 29, 2015.

(51) Int. Cl. A61K 8/891; A61K 8/25; A61K 8/26; A61K 8/29; B05B 7/14; A61K 8/85; A61K 8/02; A61K 8/64; A45D 19/02; A45D 33/02; A61Q 5/00; A61K 8/19

(52) U.S. Cl. CPC A61K 8/891; A61Q 5/00; A61K 8/25; A61K 8/26; A61K 8/29; A61K 8/19; A61K 8/85; A61K 8/027; A61K 8/645; A61K 8/022; A45D 19/02; A45D 33/02; B05B 7/1486; A61K 2800/412; A61K 2800/524; A61K 2800/43; A45D 2200/05

(57) ABSTRACT

Compositions adapted for use as a hair densifying agent are presented, as well as embodiments of a dispenser for dispensing the composition. A hair densifying system includes a dispenser adapted for dispensing a hair densifying agent with a hair densifying agent disposed therein, the system is substantially as described. The compositions can also be used to modify the color balance in a user's hair.

### CROSS-REFERENCE TO RELATED APPLICATIONS

This application is a continuation of International Patent Application No. PCT/US16/68877, filed Dec. 28, 2016, which in turn claims the benefit of priority of U.S. Provisional Patent Application Ser. No. 62/272,552, filed Dec. 29, 2015.

### BACKGROUND OF THE DISCLOSURE

1. Field of the Disclosure

The embodiments disclosed herein relates to cosmetic products, and in particular, to a hair densifying product and dispenser.

2. Description of the Related Art

Naturally thin or thinning hair is faced by many people. Unfortunately, as each of us grows older, the thinning of our hair is almost guaranteed. Individuals seeking to maintain attractive hair may indulge in hair implants, hair pieces, extensions, or other costly approaches to improve their appearance. Each is not without a drawback.

Hair implantation may not be available to an individual as it can be quite expensive and involve a lengthy process with a practitioner. Where there is only moderate hair thinning, hair pieces and extensions may not be suitable, and styling products do not provide enough of a result. None of these solutions adequately accommodate individuals with moderate hair loss or thinning, or someone with only an occasional need for appearance improvement.

Thus, what are needed are methods and apparatus that provide for hair densifying. Preferably, the methods and apparatus are simple to use and cost effective.

### SUMMARY OF THE DISCLOSURE

In one embodiment, a composition adapted for use as a hair densifying agent is provided. The composition is substantially as described within this disclosure.

In another embodiment, a dispenser adapted for dispensing a hair densifying agent is provided. The dispenser is substantially as described within this disclosure.

In a further embodiment, a hair densifying system is provided. The system includes a dispenser adapted for dispensing a hair densifying agent with a hair densifying agent disposed therein. The system is substantially as described within this disclosure.

Thus, in some implementations, a composition for densifying hair of a user is provided including silica in an amount between about 5.00 and 25.00 weight percent, fiber in an amount between about 20.00 weight percent and 50.00 weight percent including a pigment deposited thereon and/or therein, and mica in an amount between about 3.00 and 25.00 weight percent. If desired, additional pigment can be provided independently, and/or the pigment can be adhered to other components of the composition. If desired, the composition can further include titanium dioxide in an amount between about 0.10 and about 20.00 weight percent, and/or iron oxide in an amount between about 0.10 and about 10.00 weight percent. The fiber can include polyethylene terephthalate fiber. The fiber generally can have an average length between about 5 and 100 microns, for example. In some implementations, the fiber can have an average length between about 10 and 30 microns. The composition can further include oat kernel protein in an amount between about 5.00 and 35.00 weight percent, for example. The oat kernel protein can have an average particle size between about 50 and 100 microns, for example. The composition can have a bulk density between about 5.00 and about 8.00 grams per cubic inch in some implementations, and particulate in the composition can have particle sizes that span a range from about one micron to about 100 microns. In some embodiments, the composition can further include dimethicone in an amount between about 0.1 and 1.0 weight percent having a viscosity between about 5 and 100 centistoke. If desired, the composition can further include at least one preservative in an amount between 0.5 and 1.0 weight percent. The pigment causes a user's hair to appear light brown, dark brown, black, gray, blonde, red auburn, or other shades, as desired. Methods are provided herein for applying the compositions disclosed herein to hair on a user's scalp to enhance the appearance of the density of the user's hair, and if desired, to alter the apparent color or color balance of the user's hair. Moreover, treatment systems are provided herein including any of the disclosed compositions in powder form in a pump actuated spray container. The flowability of the disclosed compositions significantly facilitates their delivery when entrained in a forced gas stream.

### BRIEF DESCRIPTION OF THE DRAWINGS

The features and advantages of the disclosure are apparent from the following description taken in conjunction with the accompanying drawings in which:

FIG. 1A is a cross-sectional view of a container and dispenser in a top position;

FIG. 1B is a cross-sectional view of the container and dispenser illustrated in FIG. 1A in a bottom position;

FIG. 2A is an enlarged cross-sectional view of a portion of the dispenser illustrated in FIGS. 1A and 1B during an upstroke portion of a pump cycle;

FIG. 2B is an enlarged cross-sectional view of a portion of the container and dispenser illustrated in FIGS. 1A and 1B during a down stroke portion of a pump cycle;

FIG. 3 is an enlarged cross-sectional view of a portion of the container and dispenser illustrated in FIGS. 1A and 1B;

FIG. 4 is an enlarged cross-sectional view of a portion of the container and dispenser illustrated in FIGS. 1A and 1B;

FIG. 5A is a side view of a portion of the container and dispenser apparatus illustrated in FIGS. 1A and 1B with a nozzle member in an open position;

FIG. 5B is a side view of a portion of the container and dispenser illustrated in FIGS. 1A and 1B with the nozzle member in a closed position;

FIG. 6A is a cross-sectional view of another embodiment of the container and dispenser in a top position;

FIG. 6B is a cross-sectional view of the container and dispenser illustrated in FIG. 6A in a bottom position;

FIG. 7A is a cross-sectional view of another embodiment of the container and dispenser in a top position;

FIG. 7B is a cross-sectional view of the container and dispenser illustrated in FIG. 7A in a bottom position;

FIG. 8 is an enlarged cross-sectional view of a portion of an embodiment of the container and dispenser;

FIG. 9 depicts another embodiment including aspects of the container and dispenser apparatus;

FIG. 10 is a bottom perspective view of the dispenser nozzle of the dispenser of FIG. 9;

FIGS. 11A-11G are perspective and plan views of a first portion of an alternative embodiment of a nozzle for the dispenser of FIG. 9;

FIGS. 12A-12G are perspective and plan views of a second portion of an alternative embodiment of a nozzle for the dispenser of FIG. 9;

FIG. 13 is a perspective view of a pump for the dispenser of FIG. 9 with the nozzle components of FIGS. 11A-11G and FIGS. 12A-12G in place.

FIGS. 14-66 are photomicrographs of compositions in accordance with the present disclosure.

### DETAILED DESCRIPTION

Disclosed herein are hair densifying agents and a container/dispenser for applying the hair densifying agents. Generally, use of the hair densifying agents results in an appearance of thicker, fuller hair. The hair densifying agents may be referred to as providing volume (that is, as a “volumizing” agent). The hair densifying agents may also be referred to as “hair filler” and by other similar terms.

Embodiments of hair densifying agents disclosed herein generally include a mixture of materials. Generally, the mixture of materials is provided as a substantially dry, powdery mixture. Using the dispenser disclosed herein, or other suitable dispensers, the hair densifying agents result in a substantially even dispersion that blend well with the hair of a user. Once applied, the hair densifying agents are substantially cohesive and remain in place until washed out by the user. Generally, the hair densifying agents do not substantially flake, smear or otherwise disburse in unwanted ways.

Table 1 below provides an illustrative range for formulations of embodiments of the hair densifying agent in accordance with the disclosure. Formulas for the hair densifying agents include, among other things, a blend of minerals on and/or encapsulated within a fibrous substrate, such as a burnt clay deposited on microscopic polyester fibers. The minerals typically include silica, mica and iron oxide pigments. In combination with a natural fibrous filler, the base formula results in a free flowing powder which delivers a substantive fibrous blend that fills and thickens the hair with no or minimal aggregation. In some embodiments, the natural fibrous filler includes particulate forms of oatmeal. The levels of pigments may vary pending the shade we want to achieve.

TABLE 1
SAMPLE FORMULATION RANGES

| Ingredient | From % w/w | To % w/w |
|---|---|---|
| Polyethylene Terephthalate (“PET”) | 20.00 | 50.00 |
| Silica | 5.00 | 25.00 |
| Avena Sativa (Oat) Kernel Protein | 5.00 | 35.00 |
| Methicone | 0.01 | 0.50 |
| Diisostearyl Malate | 0.01 | 1.00 |
| Dimethicone | 0.01 | 1.00 |
| Iron Hydroxide | 0.50 | 2.00 |
| Barium Sulfate | 0.01 | 2.00 |
| Caprylyl Glycol | 0.40 | 0.60 |
| Phenoxyethanol | 0.10 | 0.20 |
| Hexylene Glycol | 0.10 | 0.20 |
| Mica | 3.00 | 25.00 |
| Red Iron Oxide CI 77491 | 0.10 | 10.00 |
| Yellow Iron Oxide CI 77492 | 0.10 | 10.00 |
| Black Iron Oxide CI 77499 | 0.10 | 10.00 |
| Titanium Dioxide CI 77891 | 0.10 | 20.00 |

*CI designates “color index”

A variety of formulations using the ingredient ranges provided in Table 1 may be realized. The resulting formulations may provide compositions suited for use on black hair, dark brown hair, medium brown hair, light brown hair and many other shades or colors of hair. Each of the ingredients can be supplied within the designated weight percentage ranges in increments as fine as 0.01 weight percent. Illustrative examples of such compositions for varying hair colors appears below in Table 2.

TABLE 2

| | | DK. | MED. | LT. |
|---|---|---|---|---|
| RAW MATERIAL | BLACK | BROWN | BROWN | BROWN |
| Polyester | 24.36 | 31.44 | 28.42 | 29.58 |
| Terephthalate | | | | |
| Silica | 9.66 | 13.47 | 12.27 | 12.73 |
| Avena Sativa (Oat) | 7.42 | 16.36 | 31.09 | 27.40 |
| Kernel Protein | | | | |
| Methicone | 0.05 | 0.05 | 0.06 | 0.09 |
| Diisostearyl Malate | 0.35 | 0.01 | 0.01 | 0.01 |
| Dimethicone | 0.35 | 0.01 | 0.01 | 0.01 |
| Iron Hydroxide | 1.68 | 1.90 | 1.72 | 1.79 |
| Barium Sulfate | 0.01 | 0.27 | 0.25 | 0.26 |
| Caprylyl Glycol | 0.50 | 0.50 | 0.50 | 0.50 |
| Phenoxyethanol | 0.15 | 0.15 | 0.15 | 0.15 |
| Hexylene Glycol | 0.15 | 0.15 | 0.15 | 0.15 |
| Mica CI 77019 | 22.00 | 11.37 | 5.25 | 5.00 |
| Red Iron Oxide CI | | 1.20 | 1.70 | 1.36 |
| 77491 | | | | |
| Yellow Iron Oxide | | 3.60 | 5.80 | 8.30 |
| CI 77492 | | | | |
| Black Iron Oxide CI | 27.00 | 11.37 | 5.25 | 5.00 |
| 77499 | | | | |
| Titanium Dioxide CI | 6.30 | 8.13 | 7.35 | 7.65 |
| 77891 | | | | |
| Biotin | 0.01 | 0.01 | 0.01 | 0.01 |
| Laurdimonium | 0.01 | 0.01 | 0.01 | 0.01 |
| Hydroxypropyl | | | | |
| Hydrolyzed Keratin | | | | |
| | 100.00 | 100.00 | 100.00 | 100.00 |

The four examples from Table 2 above were analyzed for bulk dimensional properties (average particle size within each respective mixture. The results are set forth in Table 3 below.

TABLE 3

| Particle | Gray | Lt. Brown | Med. Brown | Dark Brown | Black |
|---|---|---|---|---|---|
| Size | Sample | Sample | Sample | Sample | Sample |
| (Bulk) | 9 | 8 | 7 | 1 | 6 |
| Average* | 14.84 | 13.89 | 13.19 | 16.07 | 20.56 |
| Maximum** | 64.54 | 78.07 | 67.62 | 81.49 | 61.04 |
| Minimum*** | 0.98 | 4.22 | 3.13 | 2.53 | 3.63 |
| Std Dev | 15.57 | 11.23 | 14.53 | 14.25 | 11.81 |
| % CV**** | 104.87 | 80.90 | 110.14 | 88.68 | 57.46 |
| n = | 50 | 50 | 50 | 50 | 50 |

*The average is the mean or average size of the 50 particles for each sample. All dimensions are in microns.
**The maximum is the largest particle size measured for each sample.
***The minimum is the smallest particle size measured for each sample.
****The % CV (coefficient of variation) is the ratio of the standard deviation to the mean.

In various implementations, the disclosure provides formulations having particle sizes that span a range from about one micron to about 100 microns. In other implementations, the particle sizes range from several microns to about 60, 70 or 80 microns, as desired.

Bulk densities of the compositions vary from about 5.00 grams per cubic inch to about 8.00 grams per cubic inch, or any increment therebetween of about 0.10 grams per cubic inch. For example, the bulk density of the black formulation in the illustrated embodiment is about 4.78 grams per cubic inch, the light brown has a bulk density of about 5.35 grams per cubic inch, the dark brown has a bulk density of 6.33 grams per cubic inch, and the medium brown has a bulk density of about 7.40 grams per cubic inch. Bulk density was determined using a Scott® Paint Pigment Volumeter (obtainable from Paul N. Gardner Co., Inc., www.gardco.com). The procedure for measuring density using this equipment begins with weighing the one cubic inch square cup provided by the equipment, recording the weight, and placing the cup under the baffle box funnel, ensuring that the cup is centered. The sample to be tested is then placed on the screen, and brushed gently so that the powder flows down through the baffle box and into the cup. A steady flow of the powder is maintained to allow the powder to flow until it completely fills and overflows on all sides and corners of the cup. The cup is carefully removed, and excess powder is removed by passing a spatula across the top of the cup. The exterior surfaces of the cup are wiped using a laboratory wipe. The cup with contents is then reweighed and the weight recorded to the nearest 0.01 gram. This procedure is then repeated at least three times or until consistent numbers are obtained. The density in grams per cubic inch is then calculated by simply subtracting the weight of the container from the combined weight of the container and contents.

Synthetic fibers are such as polyester (e.g., carboxylated polyester) and nylon may be included. Natural fibers may be included, if desired, and classified according to their type. For example, vegetable, or cellulose-base, class fibers can include such fibers as cotton, flax, and jute. Animal, or protein-based fibers can be included such as wool, mohair, and silk.

In some implementations, fibers (such as PET fibers) are provided having burnt clay deposited thereon and/or therein. While any average suitable length can be used, in some implementations PET fibers can be provided having an average length between about 5 microns and about 100 microns, or any length therebetween in increments of about one micron. In a further implementation PET fibers can be provided having an average length between about 10 microns and about 30 microns. The fibers are preferably present in the formulation in an amount between about 20.00 and about 50.00 weight percent, or any amount therebetween in increments of about one weight percent.

Methicone, dimethicone and Diisostearyl Malate aid in pigment adhesion to the hair, and to make the pigments moisture-resistant. While pigment balances are set forth in the above examples for darker shades of hair, the amounts of black, red and yellow iron oxide can be adjusted to create additional shades, for example, to match shades of red hair and blonde hair, auburn hair, and the like. The amount of mica can also be adjusted to effect a change in shade.

For example, the dimethicone (polydimethylsiloxane fluid) can be provided having a viscosity between about 5 centistokes and about 350 centistokes, or any increment therebetween of about one centistoke. In some implementations, the dimethicone has a viscosity between about 5 and 15 cs, 10 and 30 cs, 20 and 50 cs, about 10 and 40 cs, and 5 cs and 100 cs, among others.

In some implementations, a natural fibrous filler can be provided that includes particulate forms of oatmeal, such as oat proteins. Such additives can have an average particle size, for example, between about 25 and about 250 microns, or any increment therebetween of about one micron. In some implementations, the fibrous filler can have an average particle size of about 25, 50, 75, 100 or 125 microns.

The mica can be provided in part as a combination such as Colorona® mica black from EMC Performance Materials in Philadelphia, Pa. that includes a mixture of iron oxide (Fe₃O₄) in a weight percentage range of about 46.0-54.0 percent (CI 77499), mica in a weight percentage range of 36.0-50.0 percent (CI 77019) and titanium dioxide in a weight percentage range of 4.0-10.0 percent (CI 77891), or in any sub-increment of 1.0 weight percent, for example.

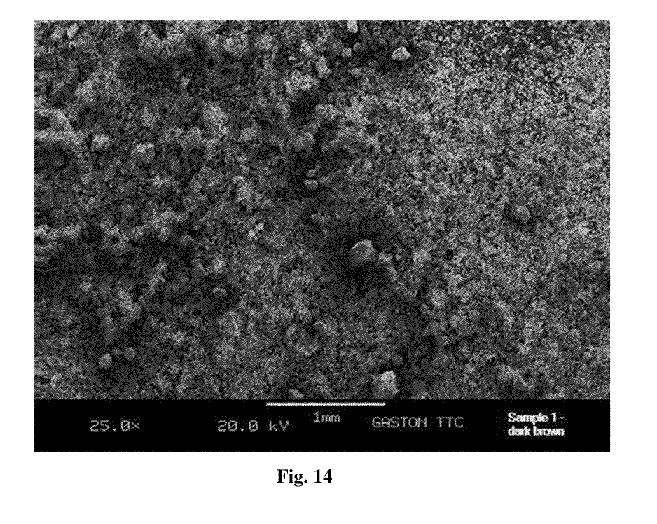

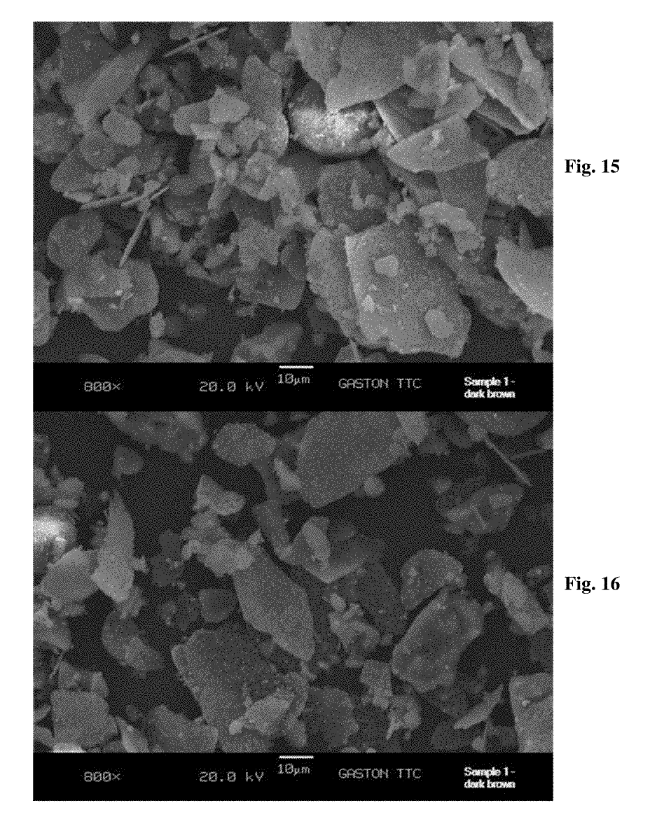

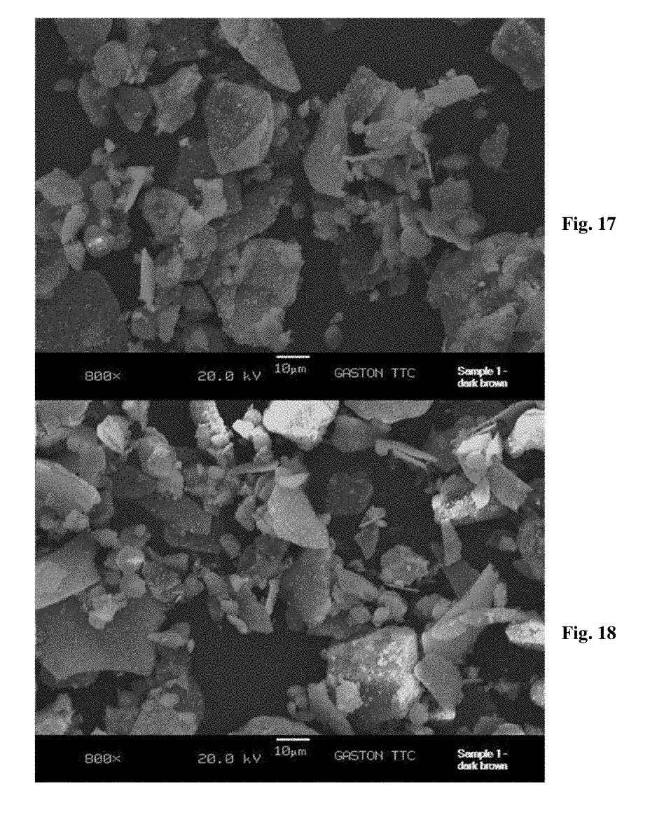

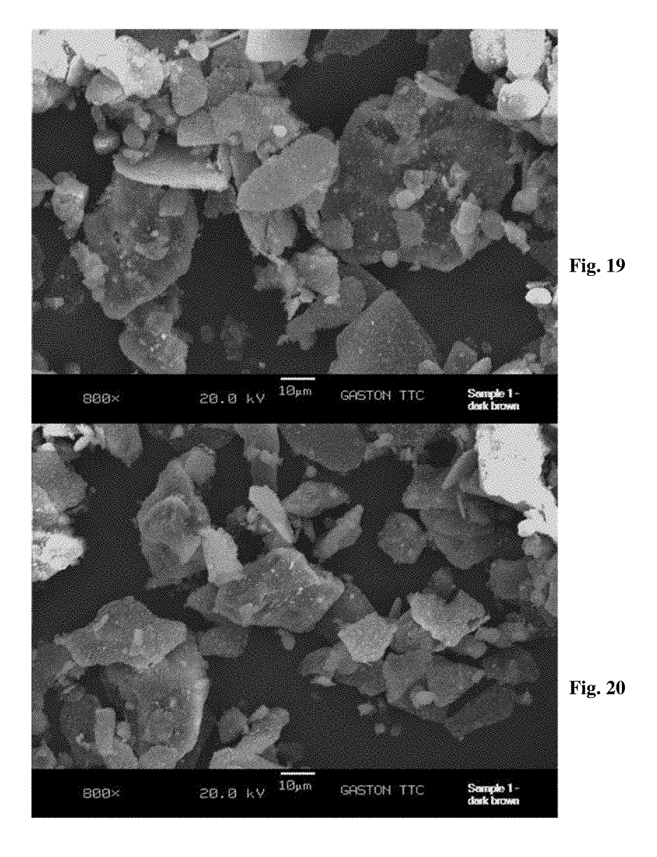

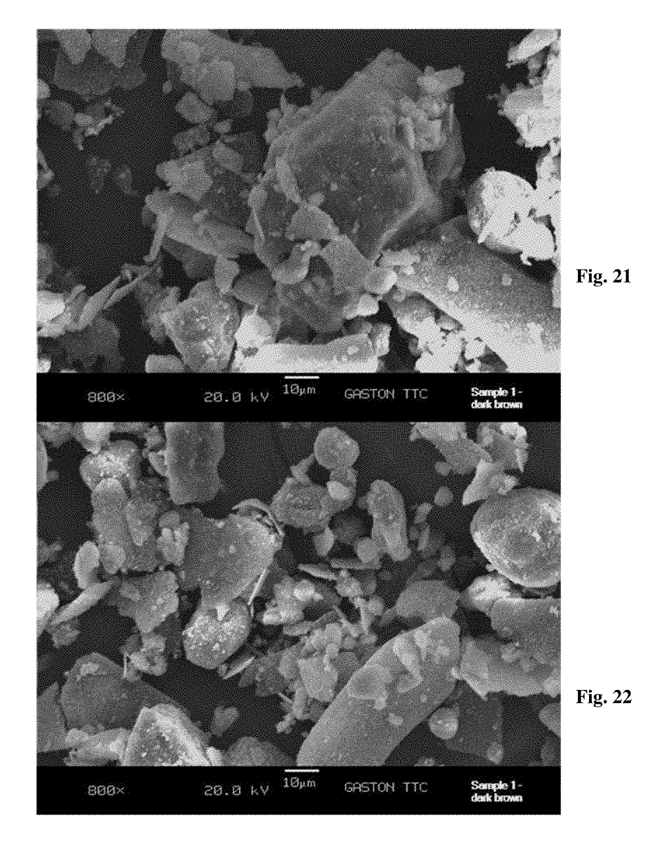

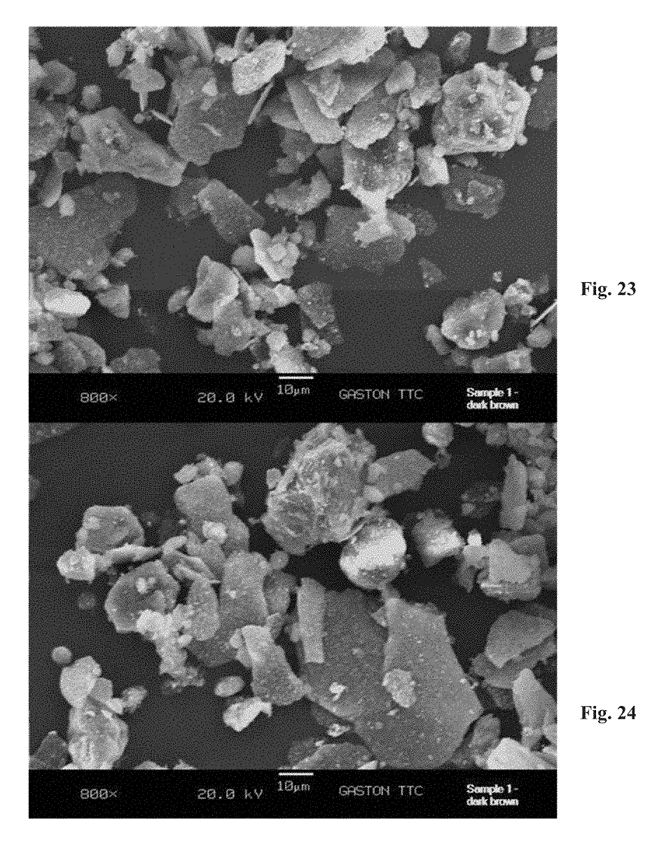

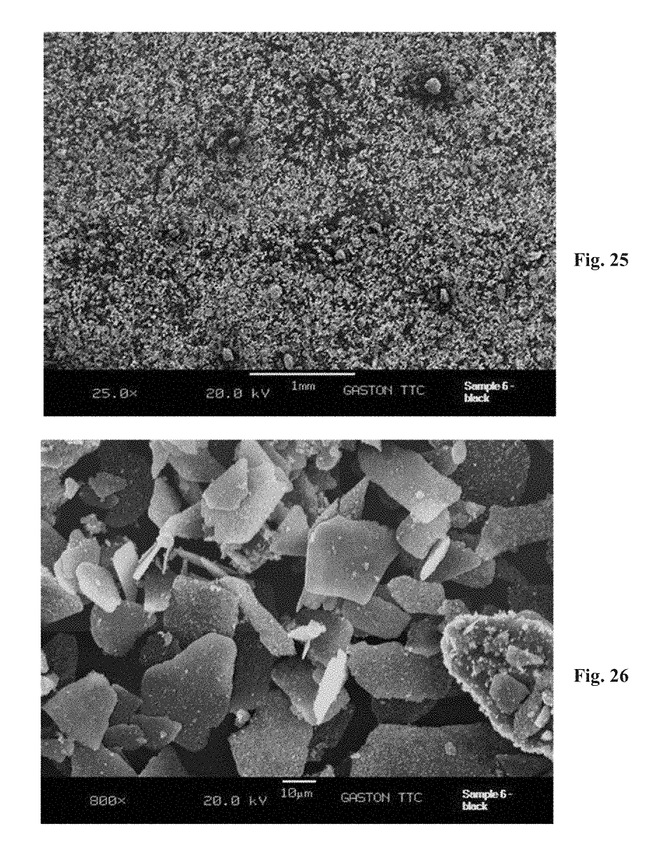

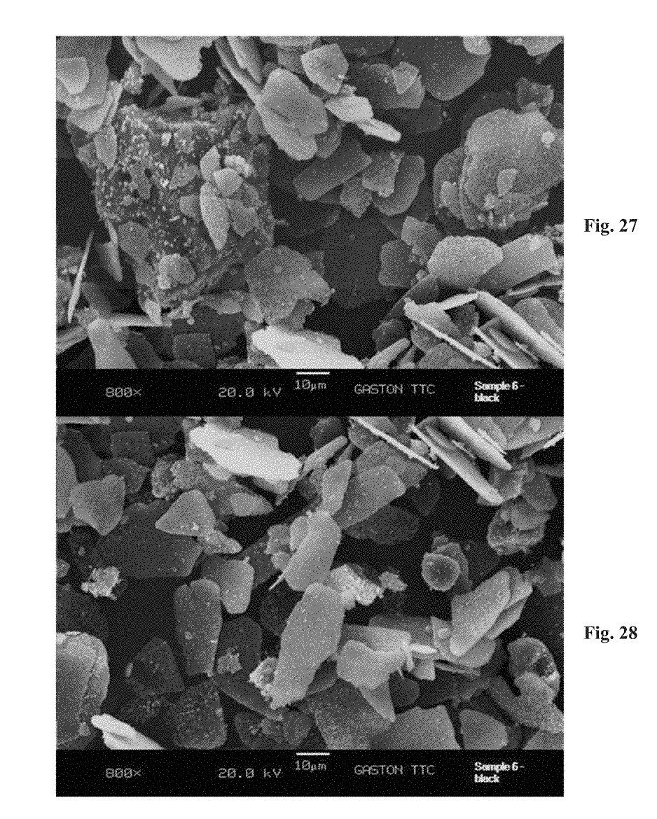

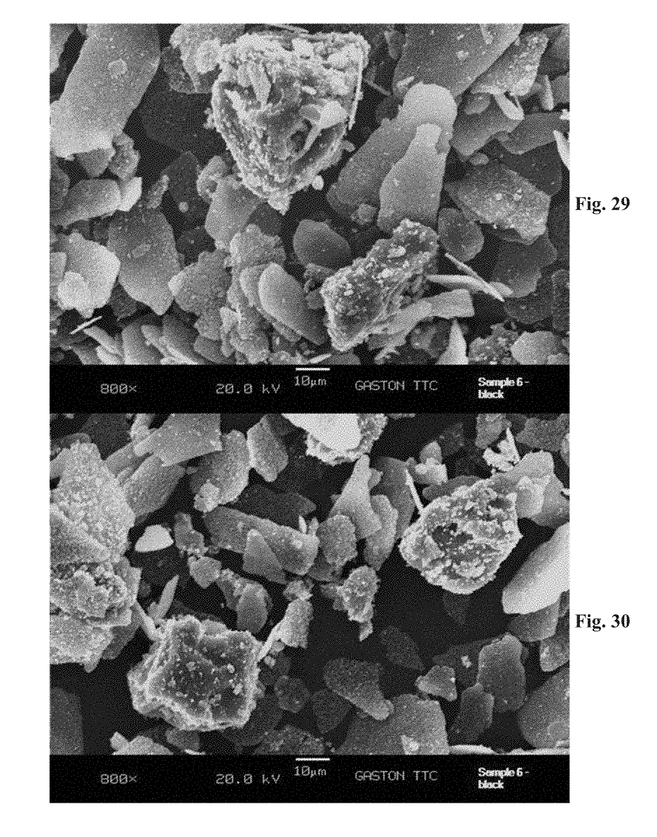

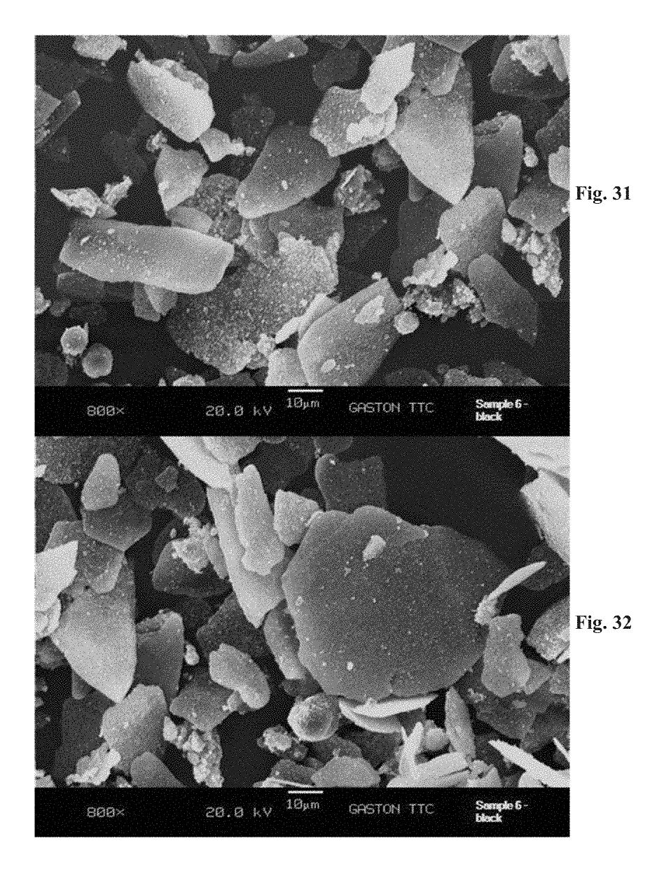

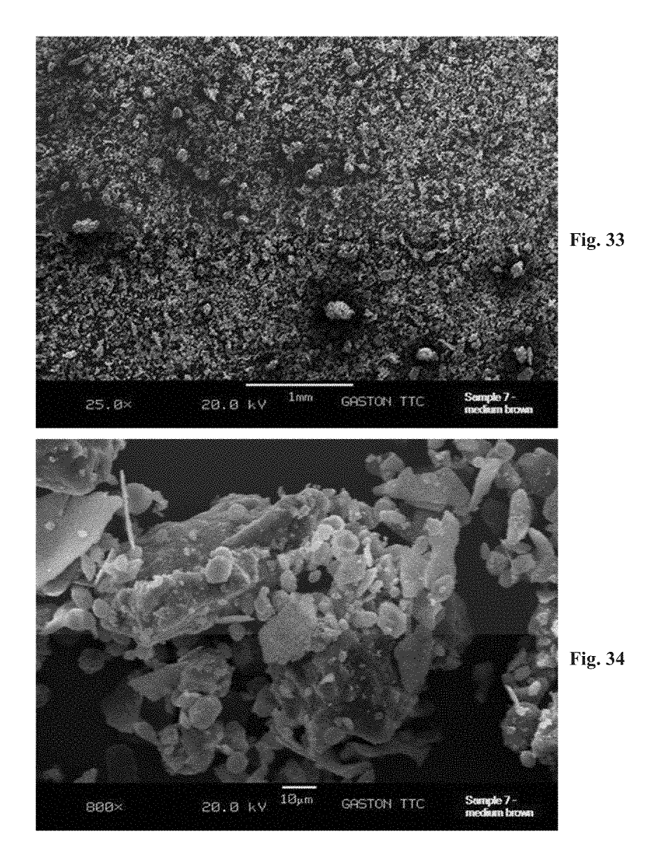

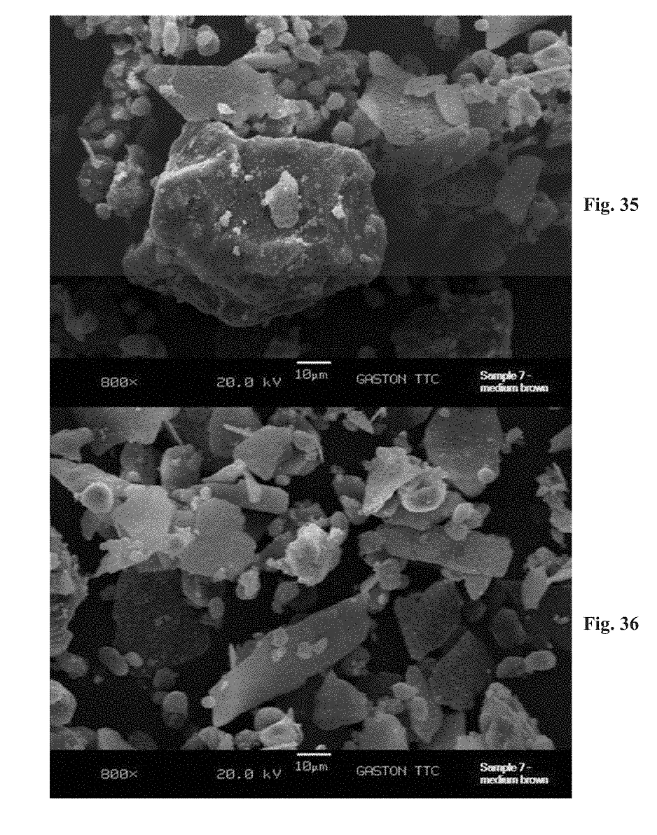

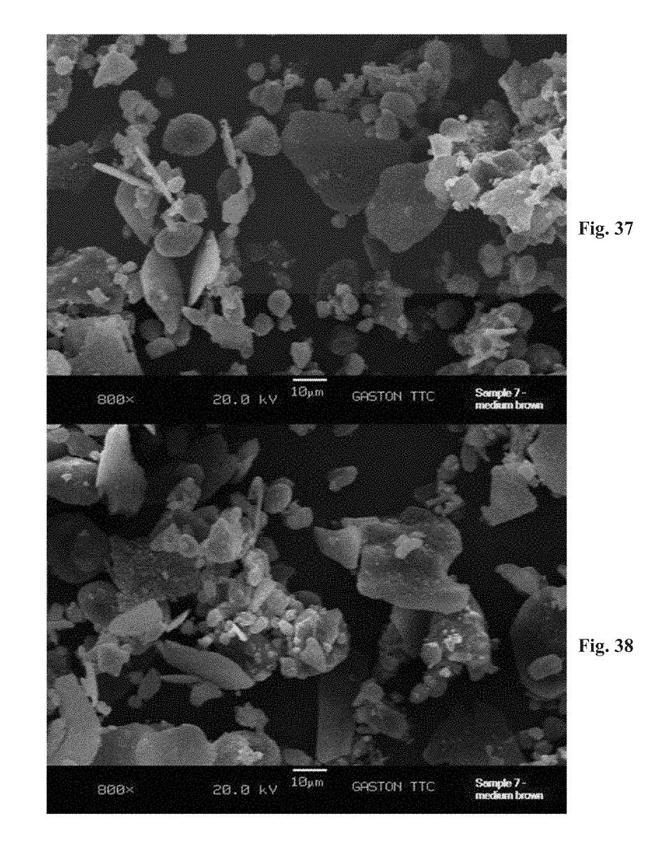

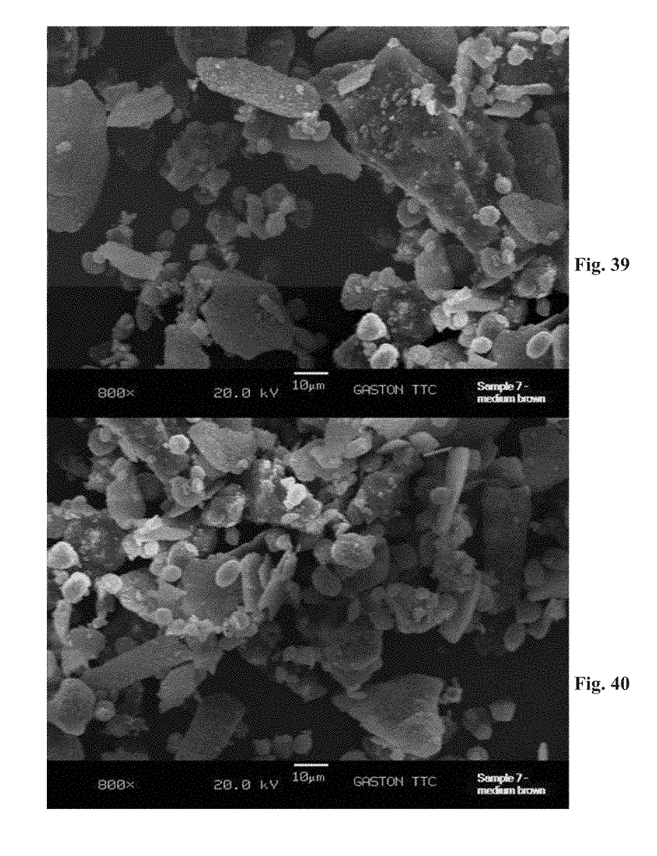

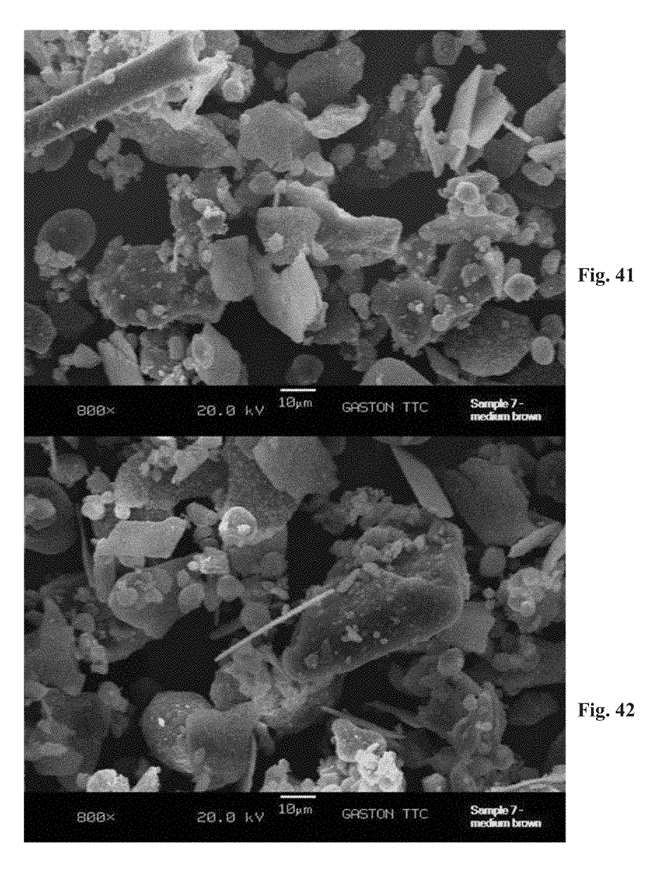

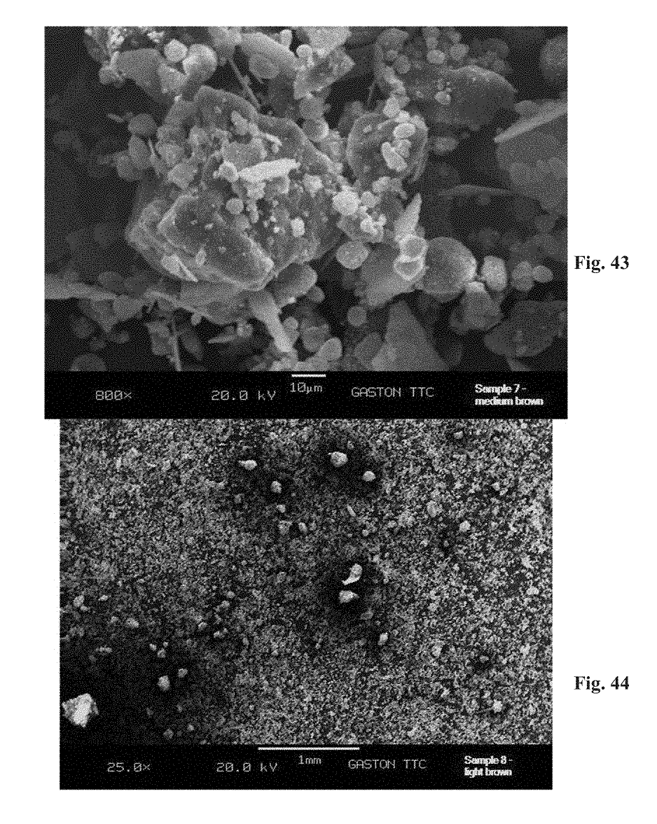

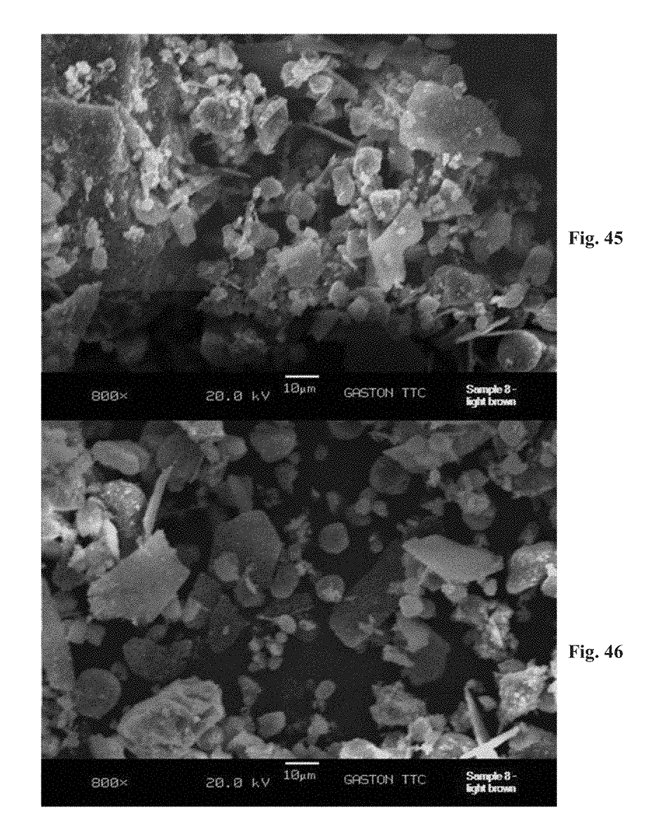

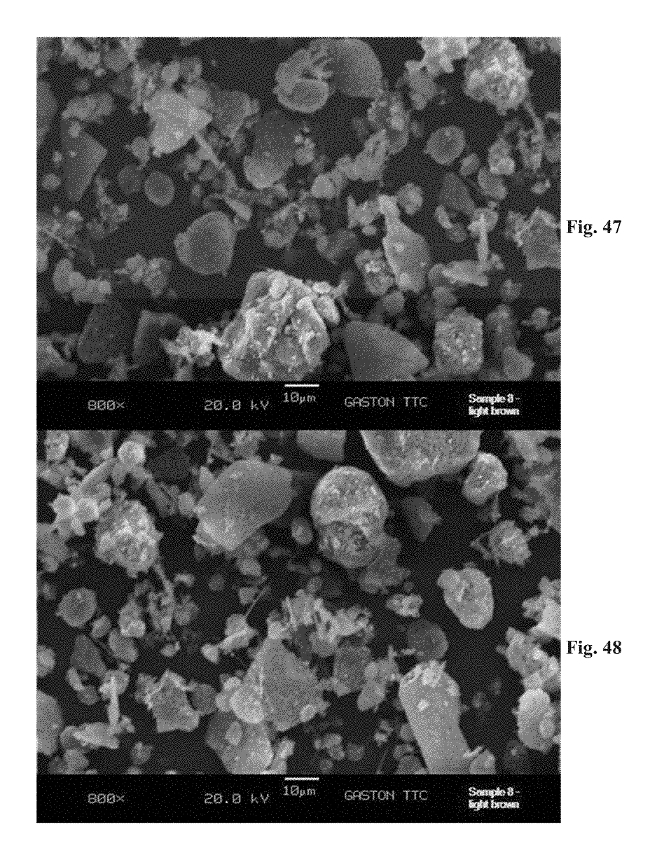

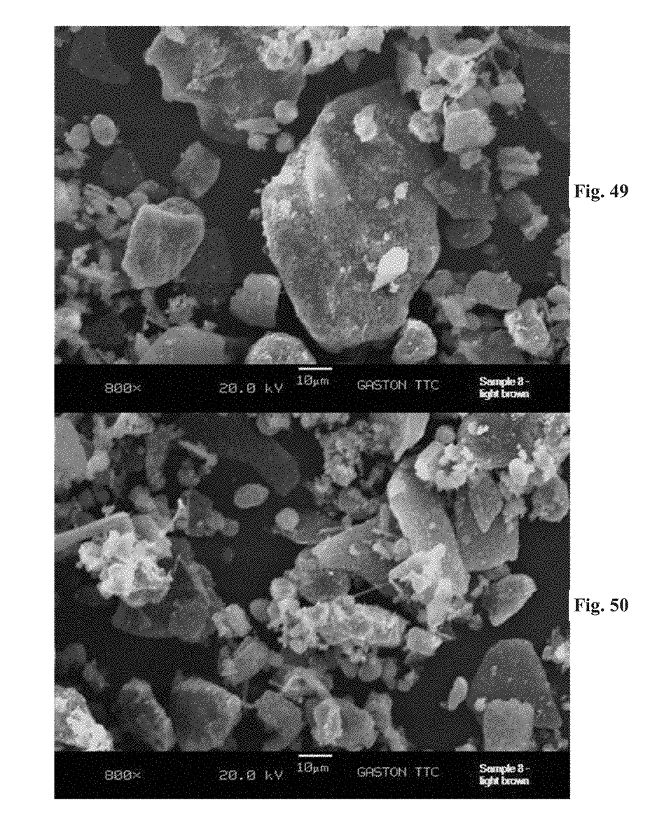

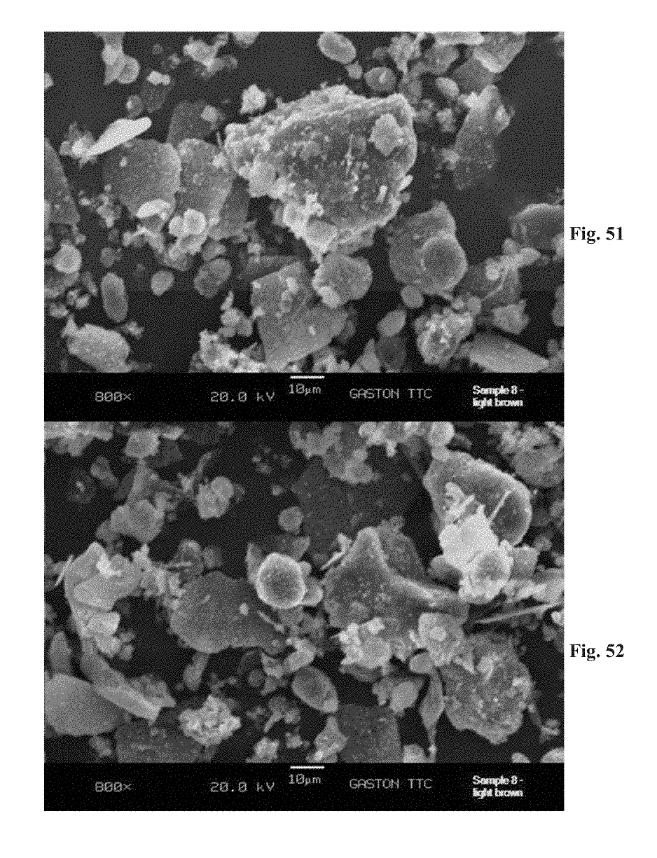

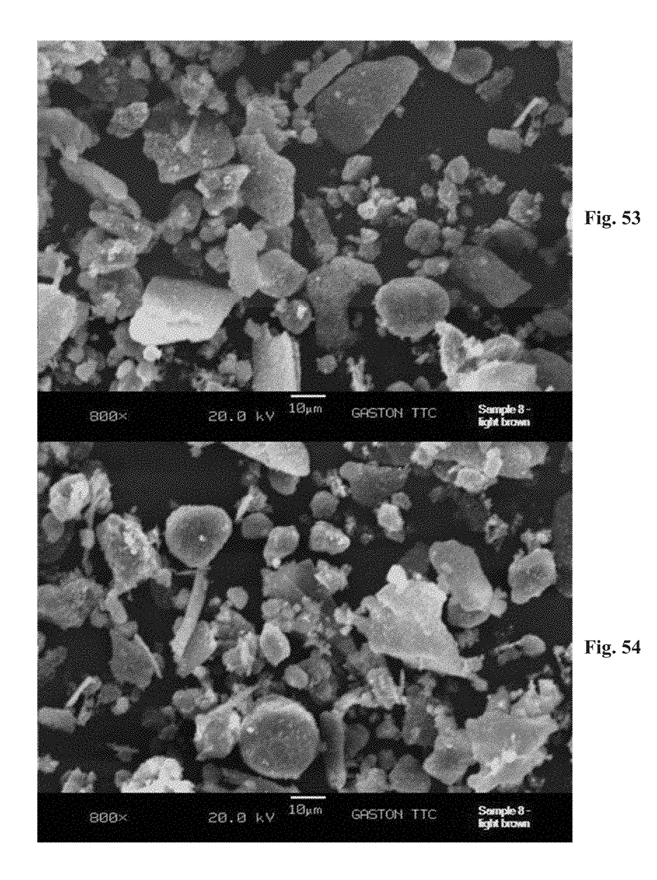

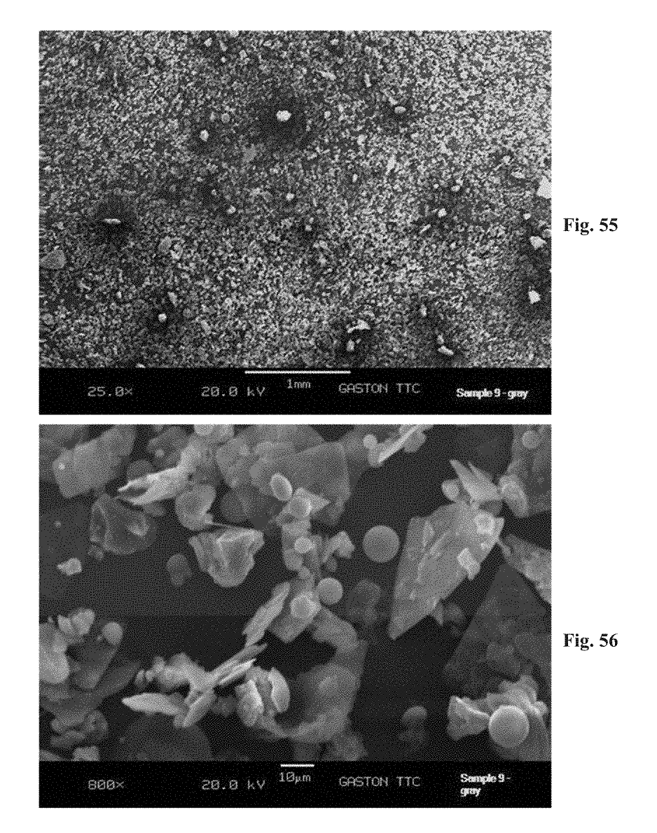

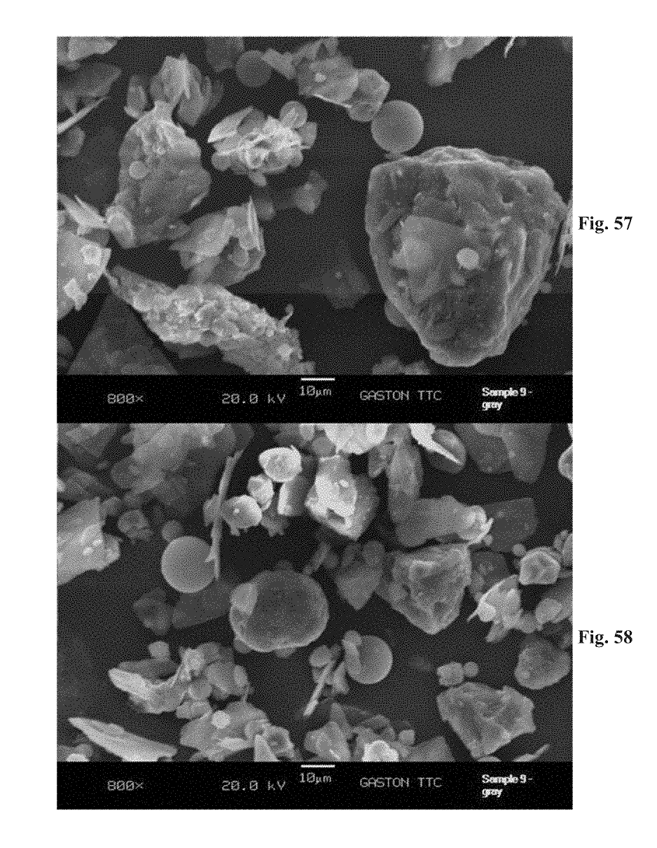

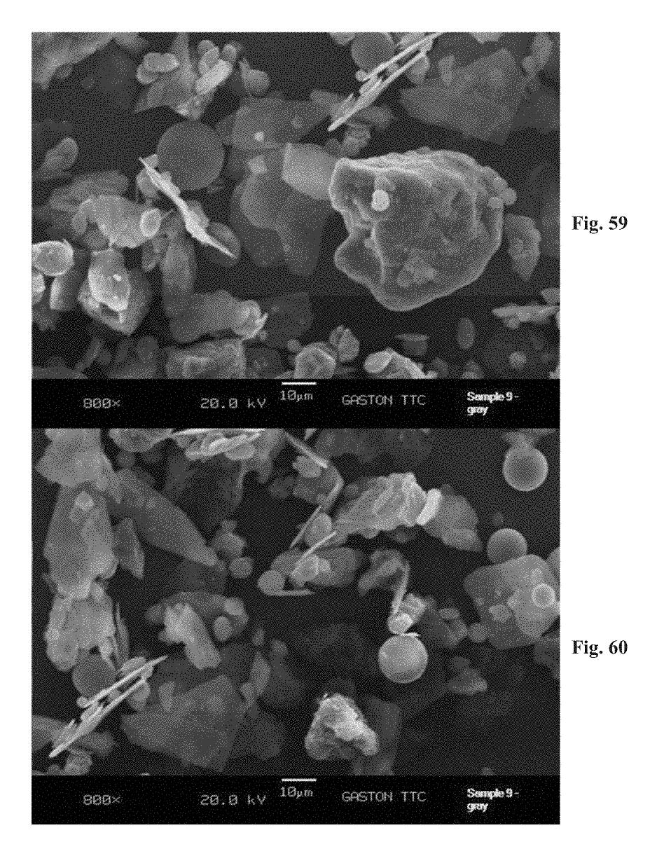

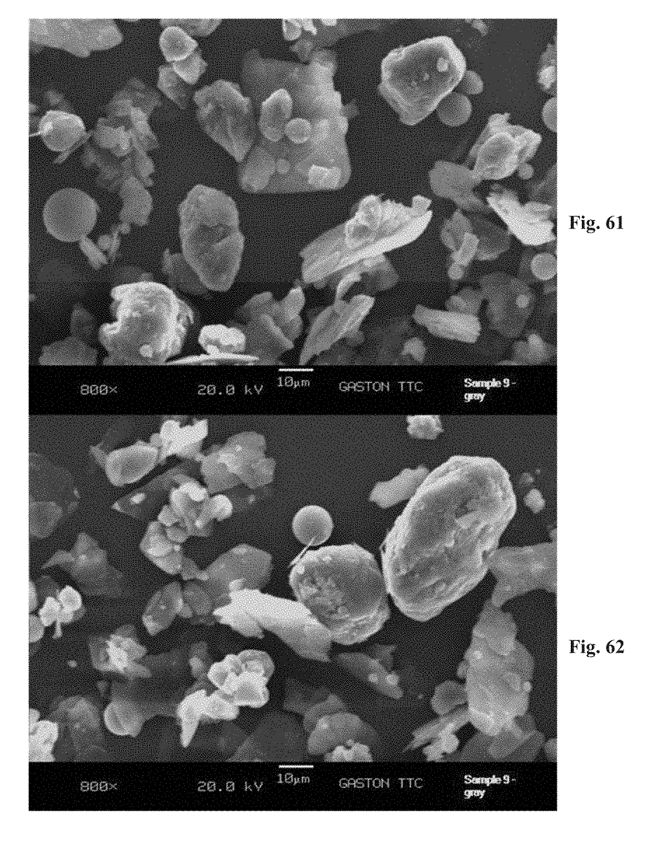

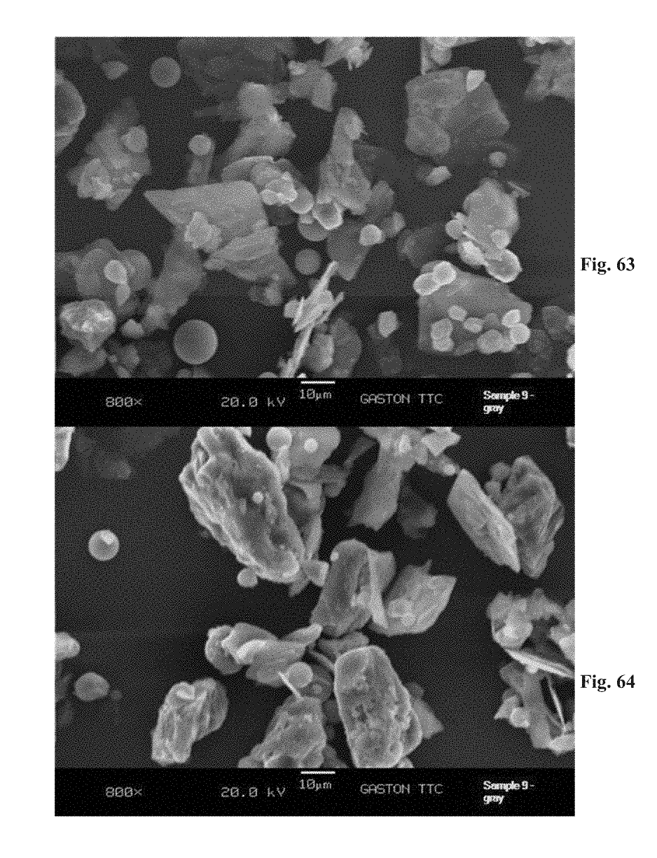

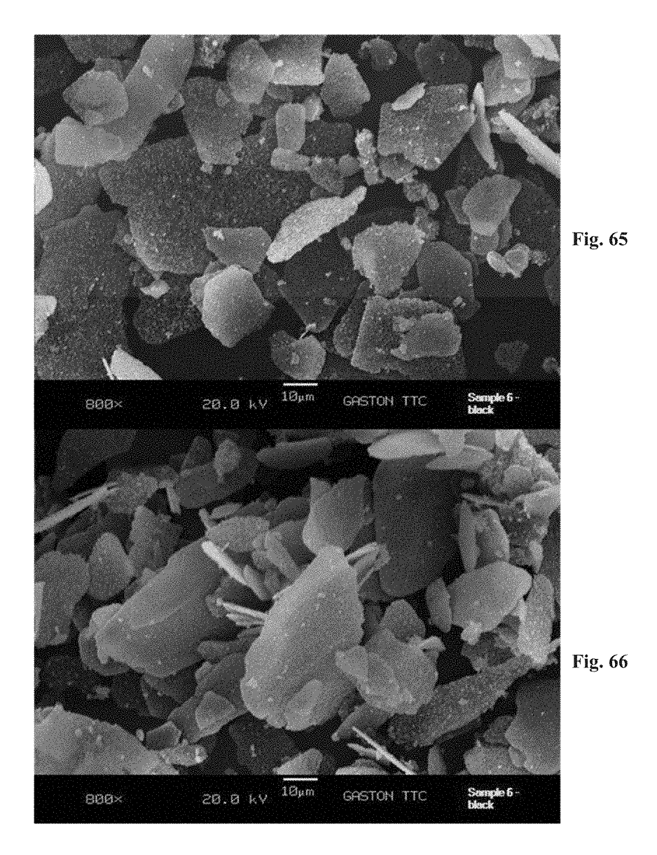

Photomicrographs of each of the samples above (i.e., Samples 1, 6, 7, 8, and 9) are provided in FIGS. 14-66 at magnifications of 25× and 800×. As can be seen, the compositions have a generally similar physical appearance. However, it will also be appreciated that the provision of adhesive agents and hydrophobic agents can act to enhance the propensity of the composition to be attracted to hair. Moreover, providing components that have a large surface area to volume ratio, and that tend to be dielectric materials, such as fibers and plates, can act to enhance adhesive bonding, as well as to enhance electromagnetic attraction by the development of electric fields thereon that tend to attract the particulate to hair fibers.

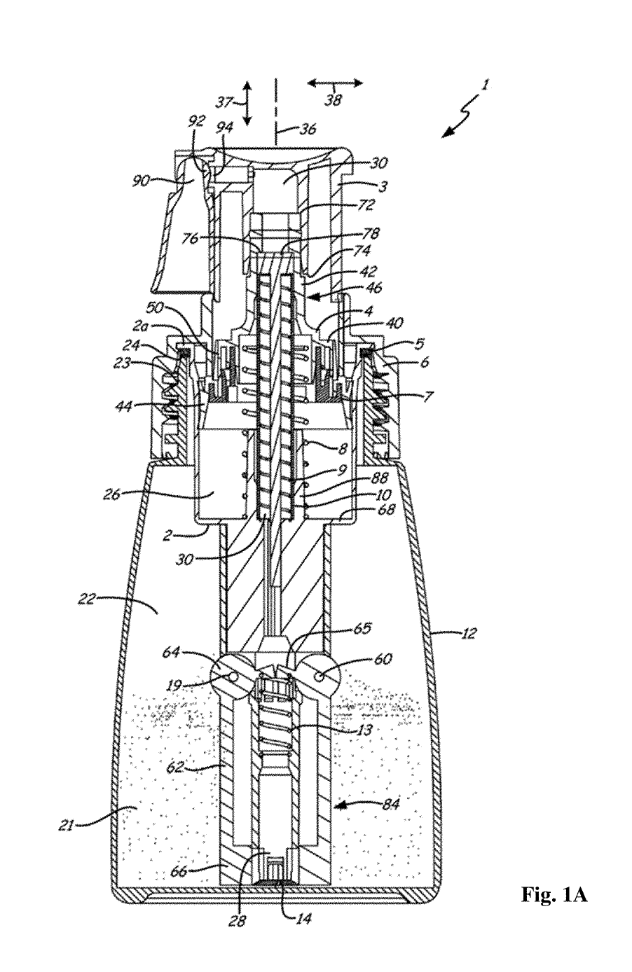

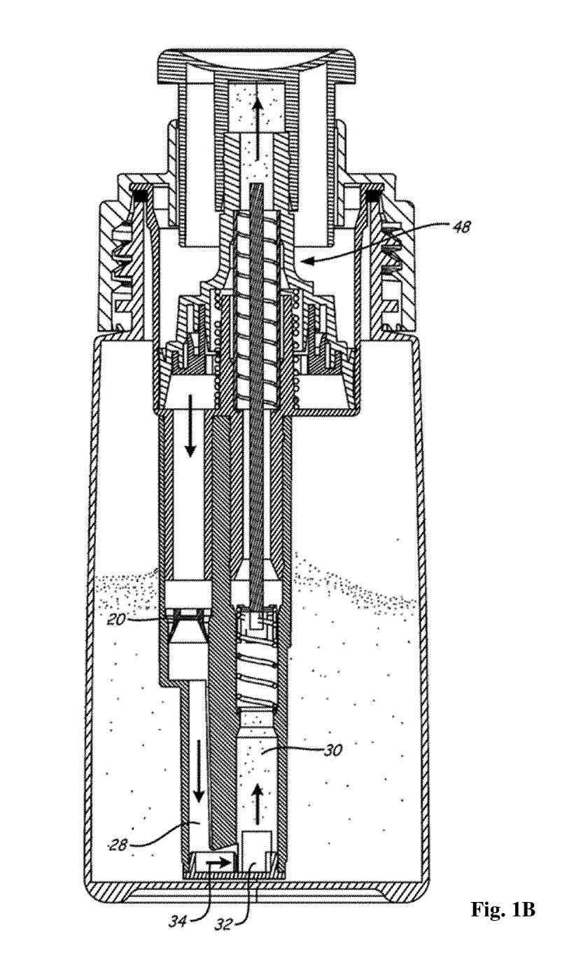

Referring to FIGS. 1A and 1B, a first embodiment of a pump dispenser 1 may be particularly suited for dispensing hair densifying agents provided in accordance with the disclosure under a manually-developed pressure. The hair densifying agent may be solid particulate matter that is dispersible into a suspension, pseudo-suspension, or mixture in air, so as to be dispensable under the pneumatic force of pressurized air as a suspension, dispersion, or mixture of solid matter in a moving air stream.

As discussed herein, the terms “top,” “bottom,” “upper” and “lower,” as well as any similar related terms are used to describe the component parts of the dispenser and their relative positions or orientation. Such terms are used only with respect to the drawings, and should not be considered limiting as to the absolute orientation of the component parts in operation.

A combination of the container and dispenser may be referred to herein simply as the “dispenser.”

Referring to FIGS. 1A and 1B, a pump dispenser 1 may be particularly suited for dispensing hair densifying agent under a manually-developed pressure. The hair densifying agent may be solid particulate matter that is dispersible into a suspension, pseudo-suspension, or mixture in air, so as to be dispensable under the pneumatic force of pressurized air as a suspension, dispersion, or mixture of solid matter in a moving air stream.

Pump dispenser 1 includes a container 12 defining a cavity 22 in which hair densifying agent 21 may be stored and prepared for dispensation. Container 12 may be in the form of, for example, a plastic bottle having a threaded neck portion 23 that cooperatively engages with a collar 6 for securement of the pumping mechanism to container 12, as will be described in greater detail herein below. Container 12 may assume a variety of configurations and materials suitable in the containment and protection of hair densifying agent 21.

Pump body 2 is positionable in container 12, and securable to neck portion 23 by being press fit between collar 6 and a sealing area 24 of neck portion 23. In the illustrated embodiment, a resilient gasket 5 assists in sealingly securing pump body flange 2a to sealing area 24 of neck portion 23. The threadable engagement of collar 6 to neck portion 23 desirably secures pump body 2 to container 12 in a sealed manner.

Pump body 2 includes a pump chamber 26, a pickup chamber 28, and an actuator chamber 30. The operation of pump dispenser 1 provides for manually pressurized air flow to pass from pump chamber 26 to pickup chamber 28, and subsequently to actuator chamber 30, and ultimately out through nozzle member 15 of actuator 3. The operation of pump dispenser 1 will be described in greater detail hereinbelow, and it is contemplated that one or more valves, pistons, and the like may separate one or more of pump chamber 26, pickup chamber 28, and actuator chamber 30. However, in the illustrated embodiment, pressurized air or other gas may be communicated by the operation of pump dispenser 1 from pump chamber 26 through pickup chamber 28 and actuator chamber 30. Accordingly, the combination of pump chamber 26, pickup chamber 28, and actuator chamber 30 may be alternatively considered as a single fluidly connected chamber. For the purposes of this description, however, portions of the pressurized air travel through pump dispenser 1 will be described as the above-indicated chamber segments. It is to be understood that no specific structure may define a transition from or between any of pump chamber 26, pickup chamber 28, and actuator chamber 30, with such chambers being delineated herein for descriptive purposes only.

In the illustrated embodiment, pump chamber 26 communicates with pickup chamber 28 through a separation valve 20, which may be a one-way valve permitting air flow into pickup chamber 28 from pump chamber 26 upon a sufficient pressure drop from pump chamber 26 to pickup chamber 28. An example separation valve 20 is a check valve with a predetermined opening force that opens only when a sufficient pressure differential is created between pump chamber 26 and pickup chamber 28. In most embodiments, the force necessary to open separation valve 20 is relatively small, in that the primary utility of separation valve 20 may be to prevent backflow of air and/or hair densifying agent 21 from pickup chamber 28 to pump chamber 26. Another utility of separation valve 20 may be to provide a “burst” airflow from pump chamber 26 to pickup chamber 28, as a sudden pressurized airflow released from pump chamber 26 upon exceeding the opening force of separation valve 20. Such a bust airflow into pickup chamber 28 may assist in dispersing hair densifying agent 21 into the moving airstream in pickup chamber 28 for passage of a hair densifying agent/airflow mixture into actuator chamber 30.

An intake opening 32 communicates cavity 22 with pickup chamber 28, and represents an opening through which hair densifying agent 21 may be imported into the air flow path for dispensation out from pump dispenser 1. Intake opening 32 may be positioned at or near base 14 of pump body 2, wherein the force of gravity will typically congregate hair densifying agent 21 near the bottom of cavity 22, in close approximation to base 14. Consequently, hair densifying agent 21 may be available for introduction through intake opening 32 until hair densifying agent 21 is nearly or completely exhausted from cavity 22. To be effective as an entry point for hair densifying agent 21, intake opening 32 is preferably appropriately sized and configured to permit an adequate loading rate of hair densifying agent 21 therethrough to accommodate dispersion of hair densifying agent into the pressurized airstream at pickup chamber 28 at a desired volumetric concentration. In some embodiments, intake opening 32 may have an opening area of between about 5-100 square millimeters, and more preferably between about 25-50 square millimeters.

A connector portion 34 may constitute a transition from pickup chamber 28 to actuator chamber 30. In some embodiments, connector portion 34 may be located at or near base 14, and directs pressurized airflow adjacently past intake opening 32 into actuator chamber 30.

Pump body 2 has a central axis 36 that defines mutually perpendicular axial and radial directions 37, 38. Pump body 2 may be arranged to facilitate pumping actuation generally along axial direction 37, though alternative arrangements are contemplated.

Pump dispenser 1 may further include a piston 4 that is slidably engaged to pump body 2 so as to selectively generate pressurized air in pump chamber 26 upon a downwardly axial movement of piston 4 with respect to pump body 2. Piston 4 includes a piston head portion 40, a piston rod portion 42, and a piston seal portion 44 extending from piston head portion 40. Piston seal portion 44 is slidably engaged with, and preferably makes an air-tight seal with side wall 18 of pump body 2 defining a portion of pump chamber 26. Axial downward motion of piston 4 into pump chamber 26, as illustrated in FIG. 1B, compresses air within pump chamber 26, correspondingly increasing the air pressure within pump chamber 26 to a level exceeding the opening force of separation valve 20, as described above. Once open, separation valve 20 permits pressurized air to flow therethrough and into pickup chamber 28 for dispersion of hair densifying agent 21 into a mixed airflow stream delivered to actuator chamber 30. The directional arrows depicted in FIG. 1B illustrate the pressurized airflow through pump chamber 26, pickup chamber 28, and actuator chamber 30. In operation, piston seal portion 44 reciprocally moves up and down in slidable engagement with side wall 18 of pump body 2, as depicted in the relationship between FIGS. 1A and 1B. Piston 4 is in a top position 46 in FIG. 1A, and a bottom position 48 in FIG. 1B. As will be described in greater detail herein below, movement of piston 4 between top position 46 and bottom position 48 drives the operation of pump dispenser 1 in collecting and dispensing the hair densifying agent 21 under pressure.

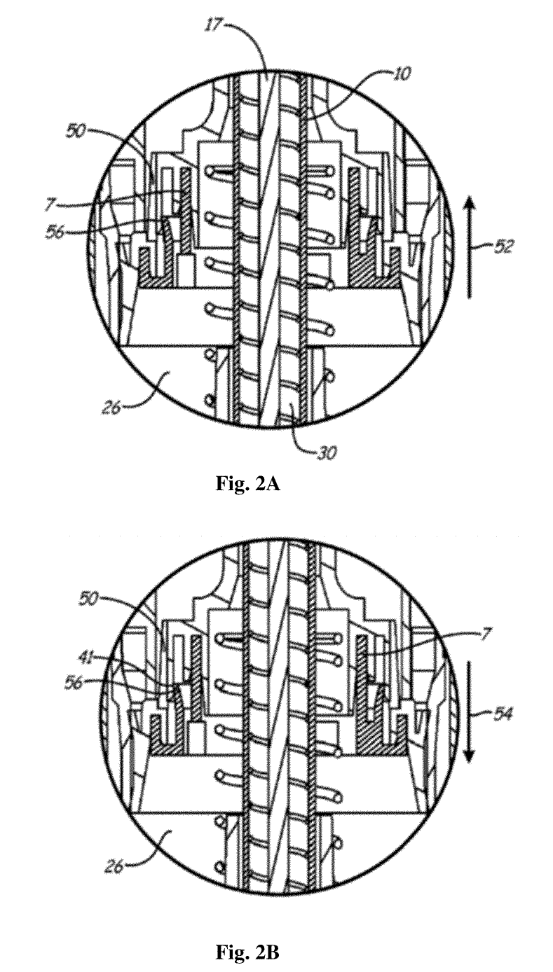

Piston head portion 40 includes an air inlet aperture 50 that is regulated by an air intake valve 7 to selectively permit passage of air from an exterior environment into pump chamber 26. An enlarged view of piston head portion 40 and air intake valve 7 is illustrated in FIGS. 2A and 2B, with FIG. 2A representing an “upstroke” of piston 4 resulting in air intake valve 7 permitting air flow into pump chamber 26, and FIG. 2B illustrating the “downstroke” of piston 4 in which air intake valve 7 prevents air from escaping from pump chamber 26 through air inlet aperture 50. The upward axial movement of piston 4 during the upstroke portion of a pumping cycle is denoted by directional arrow 52, and the axially downward motion of piston 4 in the downstroke portion of the pumping cycle being illustrated in FIG. 2B and represented by directional arrow 54. During the upstroke portion of the pumping cycle illustrated in FIG. 2A, upward axial movement of piston 4 driven by a spring bias force creates a reduced pressure environment in pump chamber 26 as the volume of pump chamber 26 expands. The reduced pressure environment within pump chamber 26 creates pressure differential with respect to the ambient, thereby developing a force that pushes valve tip 56 away from a boundary wall 41 of piston head portion 40. The displacement of valve tip 56 from boundary wall 41 is also driven by the air pressure differential between the exterior environment and the reduced pressure environment within pump chamber 26. The relative positive pressure applied against valve tip 56 displaces it away from contact with boundary wall 41. Such displacement permits the entry of air through air inlet aperture 50 and around valve tip 56 into pump chamber 26, as illustrated in FIG. 2A.

Downward axial movement of piston 4 during the downstroke of the pump cycle illustrated in FIG. 2B creates an increased pressure environment within pump chamber 26 as a result of the reduced volume within pump chamber 26. The increased pressure within pump chamber 26 forces valve tip 56 against boundary wall 41 in an air sealing manner to prevent escape of air out through air inlet aperture 50. Air intake valve 7 therefore acts as a one-way valve to permit air entry through air intake aperture 50 during the upstroke of piston 4, but prevents the escape of air from pump chamber 26 during the compression downstroke portion of the pumping cycle. Various valving arrangements for manual pumping systems are well known in the art, and a variety of configurations therefore are contemplated as being useful.

In one embodiment, one or more manipulators 19 may be pivotally secured to pump body 2 at a manipulator pivot 60. While the illustrated embodiment depicts two manipulators 19 pivotally secured to pump body 2 about respective manipulator pivots 60, it is contemplated that one or more such manipulators 19 may be incorporated with pump dispenser 1, and that various mechanisms may be employed for actuating a mechanical motion to manipulators 19. In the illustrated embodiment, manipulators 19 include a manipulator arm 62 extending from a manipulator head 64 and a distal end 66 that is configured to cooperate with intake opening 32 of pump body 2 for the passage of hair densifying agent 21 into pickup chamber 28 through intake opening 32. In some embodiments, manipulator 19 may be arranged to open and close access to intake opening 32 in sequence with the pumping cycle applied to piston 4, and to also perform a mechanical motion that aids in the distribution of hair densifying agent 21 into a relatively homogenous mass, and with a characteristic that facilitates collection and uptake through intake opening 32. For example, hair densifying agent 21 may have the tendency to settle under the force of gravity into a relatively non-flowable mass, and may further naturally settle under the force of gravity into a non-homogeneous particle size/specific weight distribution. By disturbing the mass of hair densifying agent 21 prior to loading/intake of hair densifying agent 21 to pickup chamber 28, it is more likely that a more homogeneous sample of hair densifying agent 21 may be collected for dispensation from pump dispenser 1. Manipulators 19 may therefore act as disturbing/agitating members for mixing and distributing the mass of hair densifying agent in container 12. It is also contemplated, however, that manipulators 19 may assist in loading pickup chamber 28 with a volume of hair densifying agent 21 by, in effect, “pushing” hair densifying agent 21 into pickup chamber 28 through intake opening 32. In some cases, the mechanical action of manipulators 19 may act to provide a consistent loading volume and/or mass of hair densifying agent 21 into pickup chamber 29 through intake opening 32. One aspect of the present disclosure is the enhanced ability of pump dispenser 1 to collect and dispense a known quantity of hair densifying agent 21 in each pumping cycle. Moreover, by repeatedly agitating hair densifying agent 21, manipulators 19 may aid in providing a consistent sample quality or homogeneity to pickup chamber 28 in each pumping cycle. In this manner, pump dispenser 1 advantageously is capable of dispensing a more consistent quantity and homogeneity of hair densifying agent 21 in each pumping cycle, as compared to conventional dispensing devices.

To actuate manipulators 19, an actuator rod 17 may be axially movable to actuate manipulators 19 about respective manipulator pivots 60, to thereby pivotally operate manipulator arms 62 to agitate, disperse, and collect hair densifying agent 21.

Piston 4 is axially movable with respect to pump body 2 against a first bias member 8 which, in the illustrated embodiment, is a coil spring. First bias member is placed into pump dispenser 1 under axial compression to establish a bias force urging piston 4 axially upwardly along direction 37 toward top position 46. First bias member 8 may be placed in axial compression between the pump chamber base platform 68 and a piston head platform 70. As illustrated in FIG. 1B, for example, first bias member 8 is in axially expansive contact with both base platform 68 and piston head platform 70 urging piston 4 axially upwardly with respect to pump body 2, with pump chamber base platform 68 bearing against, connected to, or integrally formed as a part of pump body 2. Actuation of piston 4 in the downstroke portion of the pump cycle must therefore overcome the bias force generated by first bias member 8.

The generation of a downward force applied against piston 4 may be originated by the user at actuator 3, wherein downward pressure upon actuator 3 is transmitted to piston rod portion 42 at cap shoulder 72 and/or cap end edge 74. Contact made between cap shoulder 72 and/or cap end edge 74 with piston rod portion 42 of piston 4 transmits the downward force applied to actuator 3 by the user to piston 4. Such downward force overcomes the bias force of first bias member 8 to move piston 4 axially downwardly in the downstroke portion of the pump cycle.

Actuator rod 17 is axially responsive to the moving force applied to piston 4 through actuator 3, as described above. The downward moving force may be applied to actuator rod 17 by piston rod portion 42 of piston rod 4 at an interface between piston rod shoulder 76 and actuator rod head 78. In the illustrated embodiment, actuator rod 17 is thereby axially movable in actuator chamber 30 as a result of the applied downward moving force from actuator 3 and piston 4.

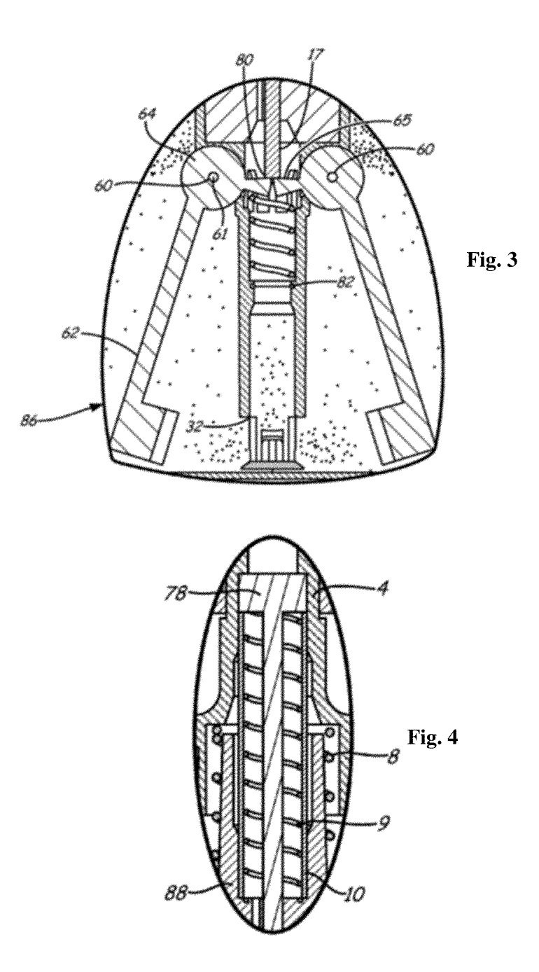

Actuator rod 17 is therefore movable axially downwardly through actuator chamber 30 to contact and actuate manipulator head 64 about its respective pivot 60. The actuation of manipulators 19 is illustrated in isolation in FIG. 3. As actuator rod 17 is axially moved downward in the downstroke as described above, end portion 80 of actuator rod 17 contacts manipulator tabs 65 extending from manipulator head 64. Continued downward movement of actuator rod 17 actuates manipulators 19 by pushing manipulator tabs 65 downwardly to cause the respective manipulator heads 64 to pivot about their respective manipulator pivots 60. Such pivoting motion, as described above, causes manipulator arms 62 to move outwardly from pump body 2 along an arcuate path about respective pivot axes 61 extending through manipulator pivots 60. In the illustrated embodiment, pivot axis 61 is substantially perpendicular to central axis 36. However, it is contemplated that other relationships may be employed to effectuate a desired movement of manipulators 19 in agitating and manipulating hair densifying agent 21 in cavity 22.

Actuation of manipulators 19 about their respective pivot axis 61 acts against a restorative force generated by a second bias member 13 which, in the illustrated embodiment, is a coil spring placed under axial compression between a pump body platform 82 and manipulator tabs 65. Downward force applied from actuator rod 17 to manipulator tabs 65 therefore acts against the restorative urging force of second bias member 13. When downward pressure against manipulator tabs 65 is released, a restorative urging force of second bias member 13 pushes manipulator tabs 65 upwardly in an opposite pivotal direction to bring manipulator arms back toward a closed position 84, as illustrated in FIG. 1A. Such a closed position orients distal ends 66 of manipulator arm 62 over intake openings 32. Thus, distal ends 66 may at least partially cover intake opening 32 when manipulator arm 19 is in closed position 82.

As described above, manipulator arms 62 are arranged to open and close in response to the pumping action of pump dispenser 1. An open position 86 of manipulators 19 is illustrated in FIG. 3. As described above, one function of manipulators 19 is to agitate and distribute hair densifying agent 21 to create a more homogenous mass of hair densifying agent 21 for entry into pickup chamber 28 through intake opening 32. It is also contemplated that the cooperation of manipulators 19 with intake opening 32 may perform one or more additional utilities for facilitating the output of consistent air/particulate mixtures. In one mode of operation, downstroke travel of piston 4 continues for a distance before actuator rod 17 comes into contact with manipulators 19. In such mode, therefore, pressurized air in pump chamber 26 is developed before manipulators 19 are actuated to move actuator arms 62 from closed position 84 to open position 86. In some embodiments, such increased air pressure in pump chamber 26 is sufficient to open separation valve 20, such that pressurized air may pass through pickup chamber 28 and into actuator chamber 30 prior to actuator rod 17 causing the opening of manipulator arms 62. In such embodiment, hair densifying agent 21 loaded into pickup chamber 28 through intake opening 32 in the previous pump cycle is picked up by the airstream moving through pickup chamber 28, and carried into actuator chamber 30 as an air, solid mixture. After the air/solid mixture has been dispensed out from actuator chamber 30, continued downward movement of actuation rod 17 presses upon manipulator tabs 65 to pivotally rotate manipulator arms 62, and to correspondingly remove distal ends 66 from a covering relationship with respect to intake opening 32. The opening motion of manipulator arms 62 distributes and, to an extent, homogenizes hair densifying agent 21 with the subsequent closing movement of manipulator arms 62 upon the release of downward pressure against manipulator tabs 65 by actuator rod 17 causing distal ends 66 to push hair densifying agent 21 into uptake chamber 28 through intake opening 32 as a loading operation for pickup by the airstream in the subsequent pump cycle. In this manner, a substantially known quantity of hair densifying agent 21 may be loaded to pickup chamber 28 in each pumping cycle through the action of the collectors formed by distal ends 66 of manipulator arms 60. It may be a desired characteristic to establish a known quantity of hair densifying agent 21 to be dispensed in each pump cycle, and the “collection” action of distal ends 66 of manipulator arms 62 to capture and collect hair densifying agent 21 in the opening and closing action cycle accomplishes the loading of a relatively consistent amount of hair densifying agent 21 into pickup chamber 28.

In another mode of operation, actuator rod 17 contacts and actuates manipulator tabs 65 to open manipulator arms 62 simultaneously with the passage of the pressurized airflow from pump chamber 26 through pickup chamber 28. In such operational mode, pressurized air developed in pump chamber 26 is able to open and pass through separation valve 20 substantially simultaneously with the opening of manipulator arms 62 from closed position 84 to open position 86. Hair densifying agent 21 is drawn into intake opening 32 by a suction force generated as a result of the pressurized airstream traveling through connector portion 34 and into actuator chamber 30. This mode of operation is illustrated in FIG. 3 by the directional arrows of hair densifying agent 21 entering pickup chamber 28 through intake opening 32.

The mode of operation of pump dispenser 1 may be driven as a result of the relationship of the length of actuator rod 17 and its position of contact in the actuation of manipulator 19 in relation to the travel distance of piston 4 between top position 46 and bottom position 48. It is to be understood that various modifications and customizations may be made for the timing and extent of opening of intake opening 32 by the actuation of manipulator arm 62 with respect to the travel of piston 4 between top position 46 and bottom position 48 in the pumping cycle.

Another aspect is illustrated in FIGS. 1A, 1B, and 4, wherein at least a portion of actuator chamber 30 is defined by a lumen of a flexible resilient member 10 that sealingly separates actuator chamber 30 from pump chamber 26 in a manner so that pump chamber 26 is fluidly communicable with actuator chamber 30 only through pickup chamber 28. Resilient tube 10 is of a characteristic which permits a sealing, air-tight connection to both piston rod portion 42 of piston 4 and support column 88 of pump body 2. Thus, a sealed passageway portion of actuator chamber 30 may be defined by resilient tube 10 between support column 88 of pump body 2 and piston rod portion 42 of piston 4. For the purposes hereof, the term “sealing” is intended to mean a substantially air-tight connection up to air pressures exerted upon components of pump dispenser 1 in its normal operation. The substantially air-tight connection forming the “sealing engagement” between resilient tube 10 and piston 4 and between resilient tube 10 and support column 88 is therefore adequate to contain and convey the mixed air/particulate solid air stream pressurized by the pumping action of piston 4 in pump chamber 26. The substantially air-tight sealing connection substantially prevents air leakage into or out from actuator chamber 30 under the normal operating conditions of pump dispenser 1.

To create the sealing connection described above, tube 10 is preferably sufficiently resilient to self-seal against the respective surfaces of piston rod portion 42 and support column 88 under a moderate radially expansive force supplied by a scaffold 9 which, in the illustrated embodiment, is a coil spring placed under radial compression in the lumen of resilient tube 10. Scaffold 9 is preferably arranged to provide a restorative radially outwardly-directed force that is sufficient to press resilient tube 10 into a sealing engagement with piston rod portion 42 of piston 4 and support column 88 of pump body 2. Scaffold 9 may further be arranged to assist in maintaining open the lumen of resilient tube 10 during the pumping cycle in which actuator rod head 78 axially compresses scaffold 9, preferably against a restorative bias force of scaffold 9, during the downstroke portion of the pump cycle. Due to the downward movement of actuator rod head 78 during the downstroke of the pump cycle, resilient tube 10 is also preferably sufficiently flexible to permit a folding or wrinkling of resilient tube 10 during the downstroke, only to be restored to its original configuration upon completion of the upstroke toward top position 46. In such a manner, resilient tube 10 forms a sealing and flexible portion of the structure defining actuator chamber 30 to accommodate the movement of actuator rod 17 through actuator chamber 30. An example material for resilient tube 10 is a silicone having an inner diameter of about 1-10 millimeters, and preferably between about 3-7 millimeters, and a wall thickness of about 0.1-4 millimeters, and more preferably between about 0.2-1.5 millimeters. Such parameters provide the desired extent of resilience and flexibility, desired for many embodiments and uses.

As described above, axial compression of scaffold 9 preferably generates a restorative axial force urging actuator rod head 78 upwardly along axial direction 37. Scaffold 9 may be a distinct component positioned in the lumen of resilient tube 10, or may instead be incorporated within or radially external to resilient tube 10. Moreover, it is contemplated that resilient tube 10 may assume configurations other than a cylindrical tube, and may have only portions of which exhibit resilient and/or flexible properties. It is to be understood that resilient tube 10 is contemplated as defining a flexible portion of the structure defining actuator chamber 30 to accommodate the movement of actuator rod 17 with respect to actuator chamber 30.

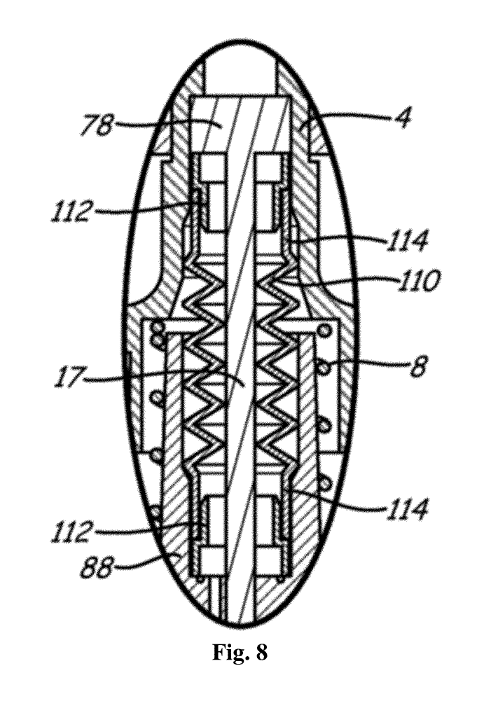

An example alternative embodiment for the combination of resilient tube 10 and scaffold 9 is illustrated in FIG. 8, wherein flexible tube 110 is engaged to actuator rod 17 through resilient plugs 112 to define a portion of actuator chamber 30, and to sealingly separate actuator chamber 30 from pump chamber 26 in a similar manner as that described above. Resilient plugs 112 may preferably have an inner diameter that is substantially equal to an outer diameter of actuator rod 17, so as to frictionally and resiliently engage an outer diameter surface of actuator rod 17, with a first resilient plug 112 being positioned at actuator rod head 78 of actuator rod 17, and a second resilient plug 112 being positioned at support column 88 of pump body 2. Resilient plugs 112 may be fabricated from a rubber or other material that exhibits elastomeric-type properties of resiliently engaging actuator rod 17 and flexible tube 110.

As illustrated in FIG. 8, flexible tube 110 may have an “accordion” type configuration to facilitate axial compression and expansion in response to the pump cycle, as described above. In one example embodiment, flexible tube 110 may be fabricated from a relatively thin-walled polyethylene, such as low density polyethylene. End sections 114 of flexible tube 110 may be frictionally engaged with resilient plug members 112, and sealingly engaged between plug members 112 and a respective one of piston 4 and support column 88.

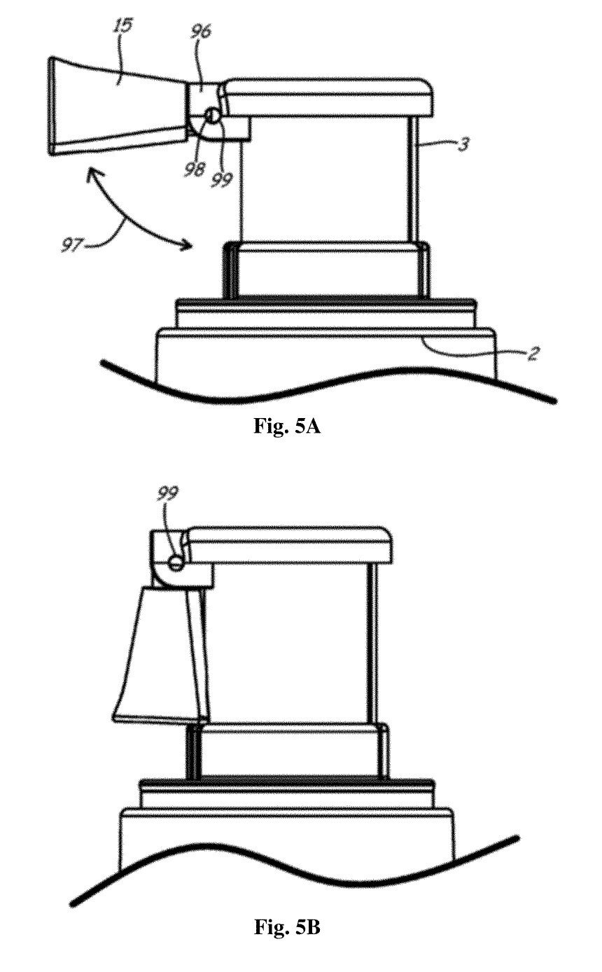

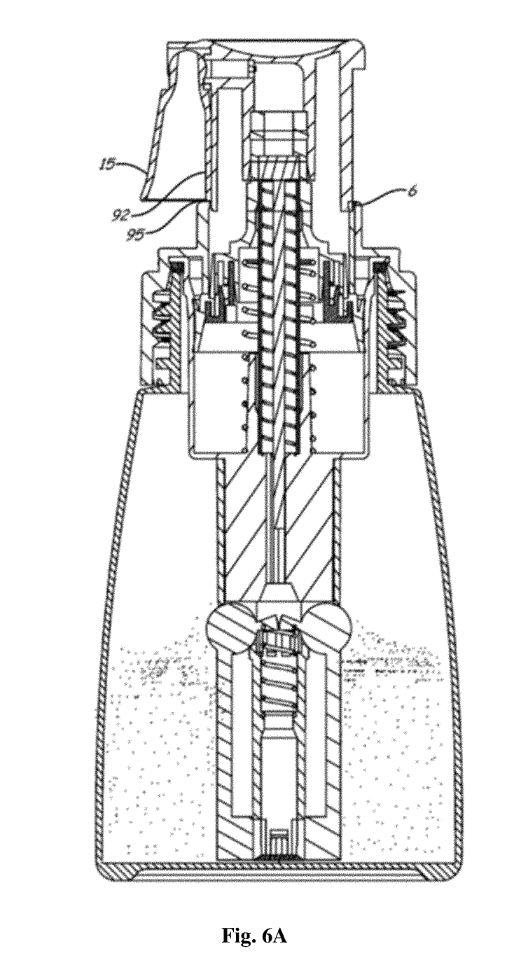

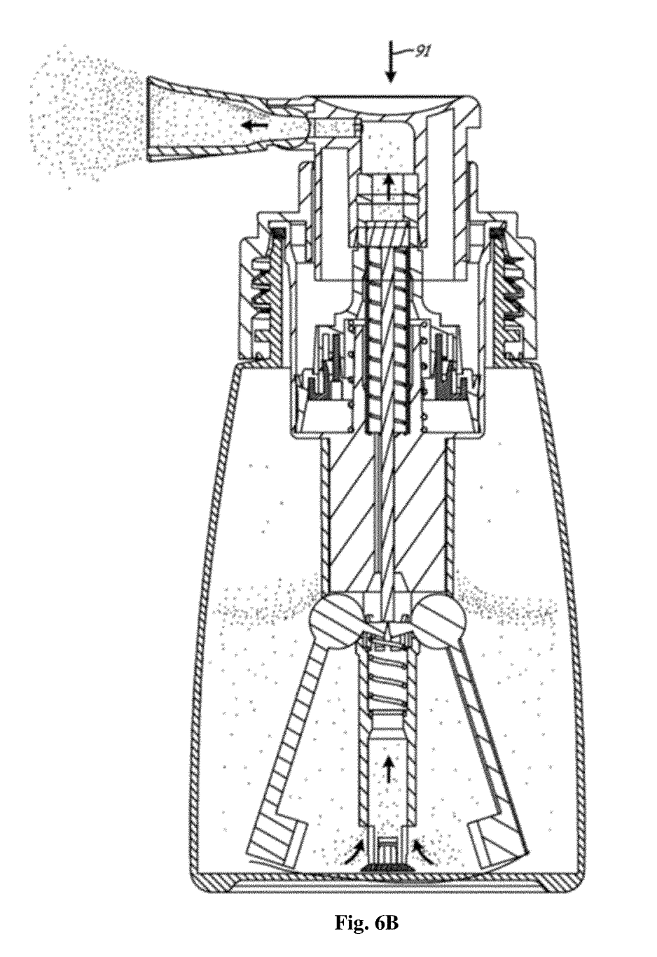

In another aspect, the pump dispenser includes a nozzle member 15 having a channel 90 extending therethrough for dispensing the air/hair densifying agent mixture out from actuator chamber 30. In the illustrated embodiment, nozzle member 15 may be selectively movable to bring channel 90 into and out from communication with actuator chamber 30 in actuator 3. In the closed condition for nozzle member 15 illustrated in FIGS. 1A, 5B, and 6A, wall 92 substantially or completely blocks an outlet 94 of actuator chamber 30 in actuator 3. When pivoted to an open condition, nozzle member 15 presents channel 90 to outlet 94 of actuator chamber 30, as illustrated in FIGS. 5A and 6B, to permit dispensation of the air/hair densifying agent flow stream out from pump dispenser 1. Nozzle member 15 may be pivotally secured to cap bracket 96 with a pivot nodule 98 extending through cap bracket recess 99. The pivoting motion of nozzle member 15 is depicted by directional arrow 97. The result is the pump dispenser 1 provides enables effortless application of the hair densifying agent by the user. That is, the user can easily and accurately direct the hair densifying agent to a target area.

An advantage introduced the pivoting nozzle member 15 is the capability to easily close off outlet 94 of actuator chamber 30, so as to inhibit or prevent moisture or other environmental element intrusion from the exterior environment into actuator chamber 30, and, more importantly, to the solids material-containing cavity 22. In some embodiments, pump dispenser 1 may be employed to operably dispense talcum powder, the physical properties of which may be significantly altered in high moisture environments. Therefore, it may be a useful function of pump dispenser 1 to limit the accessibility of moisture to hair densifying agent 21 in container 12. The capability of nozzle member 15 to pivot into a positioning in which wall 92 closes outlet 94 of actuator chamber 30 creates a closed environment for hair densifying agent 21 that minimizes moisture or other environmental element intrusion to cavity 22.

Another use of pivotable nozzle member 15 is illustrated in FIGS. 6A and 6B, wherein wall 92 of nozzle member 15 may be pivoted into a closed condition to prevent actuation of pump dispenser 1. In particular, the close condition of nozzle member 15 positions wall 92 for contact with upper surface 95 of pump collar 6. In the event an attempt is made to push actuator 3 downwardly, as in the downstroke of a pump cycle, contact between wall 92 and upper surface 95 of collar 6 prevents or stops downward motion of actuator 3. In some embodiments, the configuration of nozzle member 15 places wall 92 substantially in contact with upper surface 95 of collar 6 when piston 4 is in top position 46. Such an arrangement establishes a “lock,” in which nozzle member 15 prevents downward motion of actuator 3 when nozzle member 15 is in a closed condition. In other embodiments, however, the closed condition of nozzle member 15 permits some downward motion of actuator 3, but arrests such downward motion between top position 46 and bottom position 48. FIG. 6B illustrates nozzle member 15 in an open condition with channel 90 in fluid communication with outlet 94 of actuator chamber 30 in actuator 3. The positioning of nozzle member 15 in an open condition, as illustrated in FIG. 6B, permits downward movement of actuator 3, as depicted by directional arrow 91, in the downstroke of the pump cycle to create a pressurized air stream to dispense the air/product mixture out from nozzle member 15.

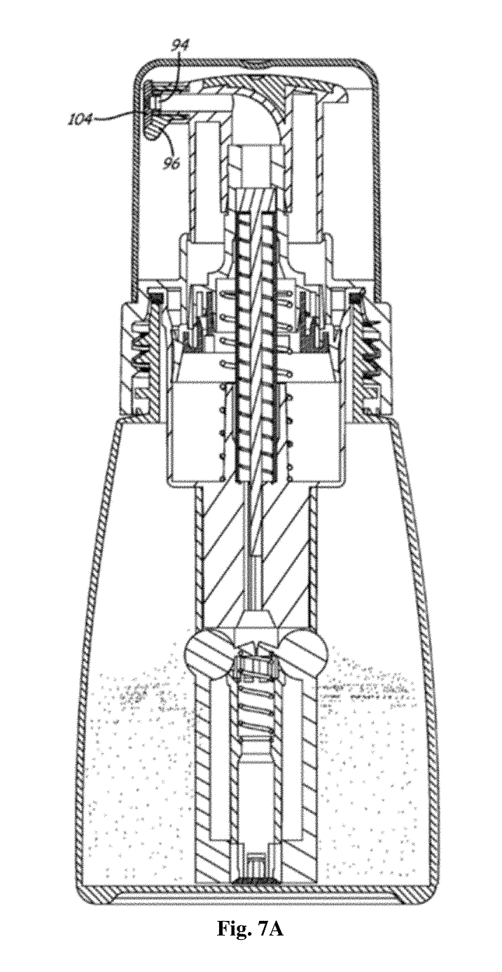

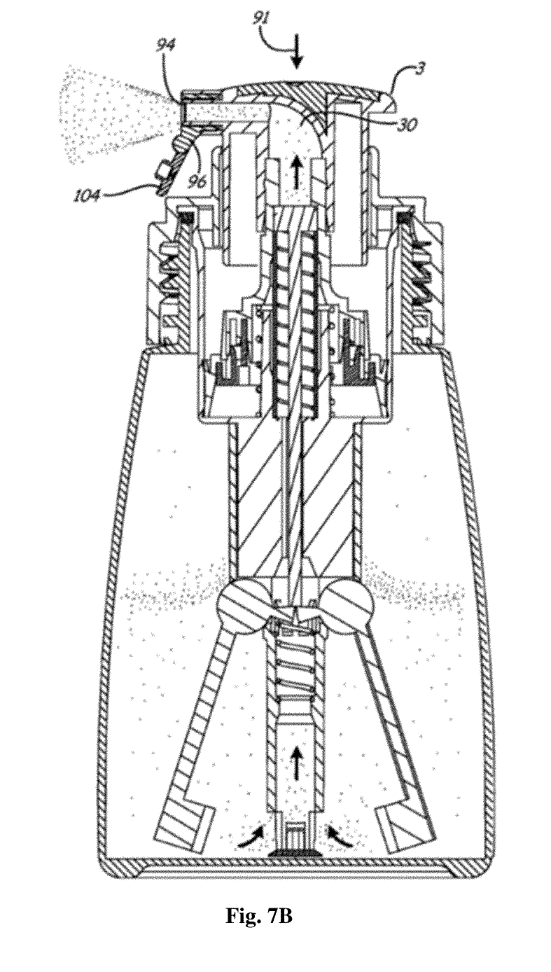

Another embodiment of the disclosure is illustrated in FIGS. 7A and 7B, wherein a nozzle cap 104 may be pivotally secured to cap bracket 96 to selectively open and close outlet 94 of actuator chamber 30 at actuator 3.

In addition to the foregoing, the operation of pump dispenser 1 is described with reference to the drawings. Initially, nozzle member 15 is rotated along direction 97 from a closed condition to an open condition to permit downward movement of actuator 3, and to present channel 90 into communication with actuator chamber 30 at actuator 3. When a downward force along directional arrow 91 is placed upon actuator 3, such force is transmitted by cap shoulder 72 to piston head platform 70 to thereby transfer the downward axial motion along directional arrow 37 to piston 4. Such downward motion is also transmitted from piston rod shoulder 76 to actuator rod head 78 so that actuator rod 17 also proceeds axially downward along directional arrow 37.

As piston 4 proceeds axially downwardly in the pump downstroke, air pressure in pump chamber 26 increases to a point at which separation valve 20 opens to permit the passage of air into pickup chamber 28. As piston 4 and actuator rod 17 continue downward movement, actuator rod end portion 80 comes into contact with, and pushes manipulator tab 65 downwardly to cause manipulators 19 to pivot about manipulator pivot 60. As manipulators 19 operate, an amount of hair densifying agent 21 enters into pickup chamber 28 through intake opening 32, wherein the pressurized air stream motivates the hair densifying agent into a mixed air/hair densifying agent flow stream into actuator chamber 30. Continued air pressure forces the flow stream mixture out through outlet 94 and channel 90 of nozzle member 15, as illustrated in FIG. 6B. Removal of the force upon actuator 3 permits first and second bias members 8, 13, and scaffold 9, to restoratively urge piston 4, actuator rod 17, and manipulator tabs 65 upwardly to place manipulator arm distal end 66 in a covering relationship with intake opening 32, and to urge piston 4 and actuator rod 17 toward to position 46. The negative air pressure created in pump chamber 26 as a result of the expanding volume in pump chamber 26 forces open air intake valve 7 to permit external environment air to enter into pump chamber 26 to substantially equalize internal and external pressures. As piston 4 and actuator rod 17 reach top position 46, pump dispenser 1 is ready for a subsequent pumping action.

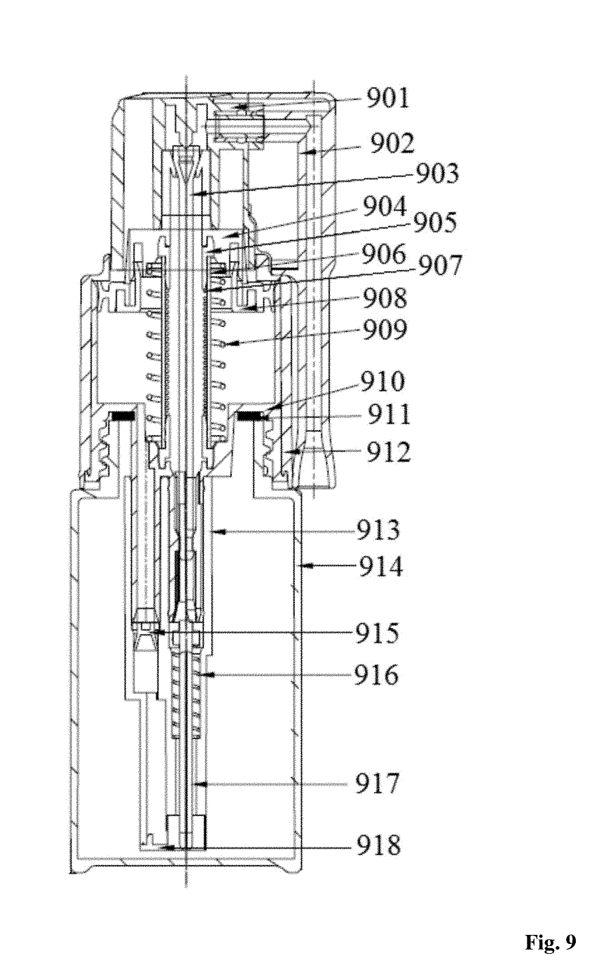

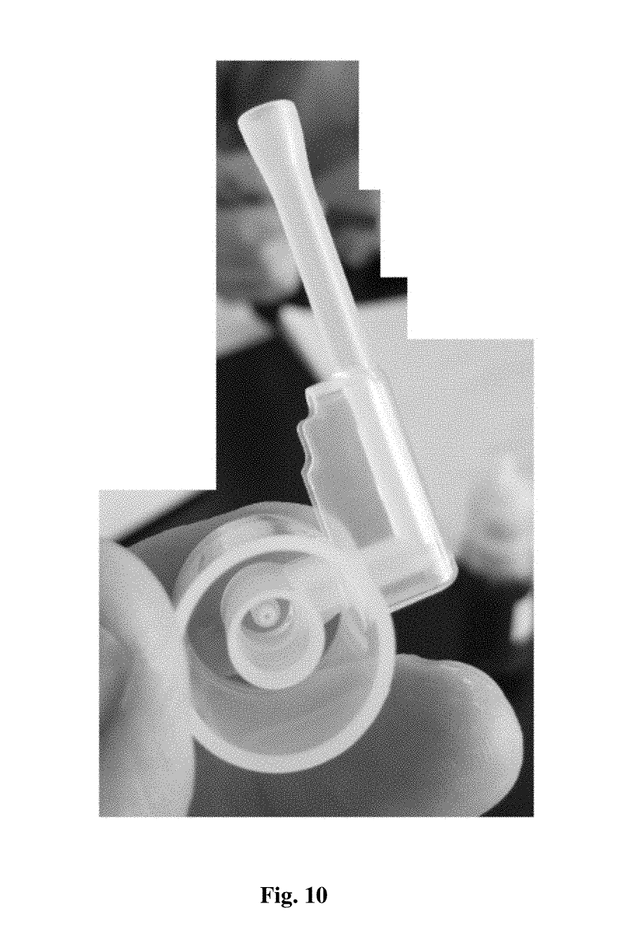

FIG. 9 is a cross-sectional view of another embodiment of a dispenser in accordance with the disclosure. As depicted, the dispenser includes a nozzle 902 that is rotatably coupled to an actuator 901 that a user depresses with a digit. The assembly of nozzle 902 and actuator 901 are presented in FIG. 10 wherein the nozzle has been rotated to be parallel with a top of the actuator. In FIG. 9, the nozzle 902 is rotated into a downward position. As is visible in the cross section of FIG. 9, the nozzle 902 defines a flow path from the actuator 901 having a first straight portion that travels to the right, as illustrated, which is connected to a second downward channel that is connected to a diffuser at the end of the nozzle. Push pole 903 is attached to actuator 901 at an upper end thereof. Actuator 901 is further configured to bear down on a piston 904 connected to a spinner 908 that is configured to slide within a cylinder, or main body 910. A gasket 911 is interposed between main body 910 and bottle portion 914, wherein bottle portion 914 includes the composition to be dispensed onto a user's hair to densify it. Plug 905 is fit into an elastic tube 906 that on an inner surface acts as a guide for spring 907, and also acts as a guide for spring 909 on an outer surface thereof. Main body 910 is surrounded and received by a housing 912. Cylinder mechanism 913 is provided that houses valve 915 as well as spring 916 and mechanism arm 917 and cover portion 918. In operation, the dispenser of FIGS. 9-10 operates similarly to the prior embodiments in that actuator 901 is depressed, having the net effect of flushing air into the bottle portion causing it to entrain particulate therein and flow up through the central flow channels of the dispenser, and out through the nozzle.

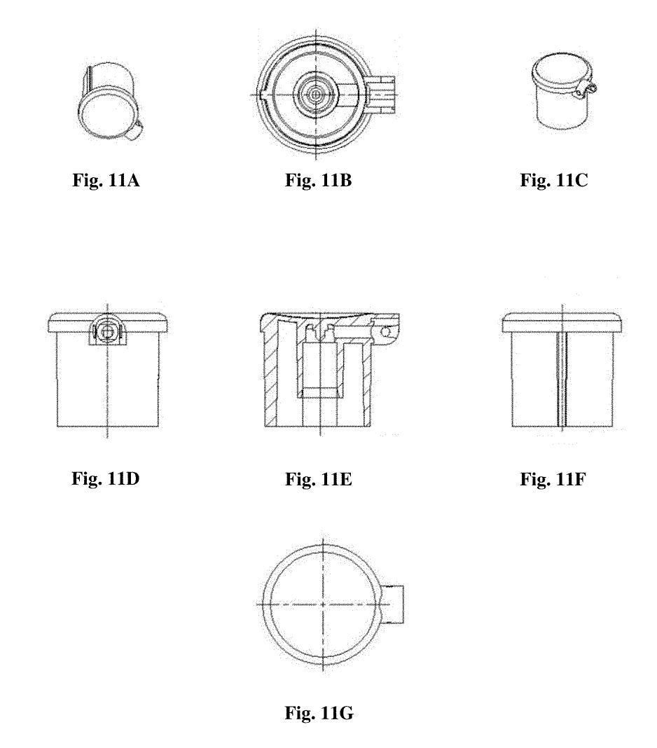

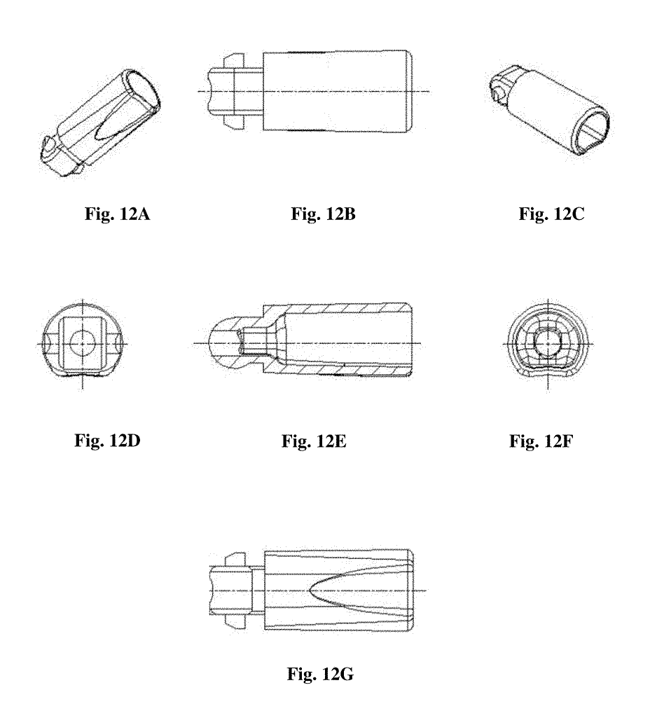

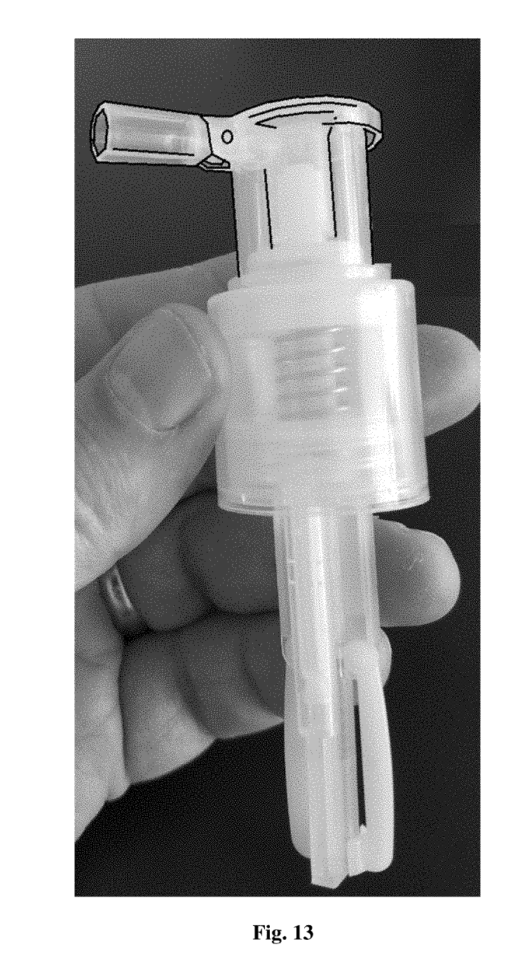

FIGS. 11A-11G present top rear perspective, top plan, top front perspective, front plan, side cross section, rear plan and bottom views of a further embodiment of an actuator that can be used in place of actuator 901. FIGS. 12A-12G present top rear perspective, top plan, top front perspective, front plan, side cross section, rear plan and bottom views of a further embodiment of a nozzle that can be used in place of nozzle 902. Nozzle 902 is pivotally attached to actuator 901, permitting the flow of air and particulate to be directed, as desired. This embodiment differs most significantly from the embodiment of FIGS. 9-10 in that it has a shorter flow path and eliminates one of the 90 degree bends of the flow path.

Having thus introduced embodiments of the hair densifying agent and embodiments of dispensers therefore, some additional aspects are now presented.

In practice, a variety of dimensions may be selected for the pump dispenser 1, and the components thereof. Among other things, careful design and construction of the pump dispenser will ultimately provide for delivery of a desired amount of hair thickening agent. In some embodiments, each pump of the pump dispenser 1 delivers about 0.02 grams to 0.05 grams of hair densifying agent 21 to a target area. Of course, the design of the pump dispenser 1 may be modified as desired to deliver other ranges of amounts of product. In one embodiment, the pump dispenser 1 includes a long targeting nozzle to enhance product placement. In short, the pump dispenser 1 may be designed as deemed appropriate to enhance manufacture and/or operation as deemed appropriate.

In some embodiments, the hair densifying agent may be dispensed using other techniques. For example, in some embodiments, the hair densifying agent may be disposed in a pressurized canister with an appropriate nozzle. In some other embodiments, the hair densifying agent may be applied with a shaker (to shake the hair densifying agent into or onto the hair), by manual application (such as being dabbed on by a hair dresser), or by any other process deemed appropriate.

Various other components may be included and called upon for providing for aspects of the teachings herein. For example, additional materials, combinations of materials and/or omission of materials may be used to provide for added embodiments that are within the scope of the teachings herein.

A variety of modifications of the teachings herein may be realized. Generally, modifications may be designed according to the needs of a user, designer, manufacturer or other similarly interested party. The modifications may be intended to meet a particular standard of performance considered important by that party.

When introducing elements of the present disclosure or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. As used herein, the term “exemplary” is not intended to imply a superlative example. Rather, “exemplary” refers to an embodiment that is one of many possible embodiments.

While the disclosure has been described with reference to exemplary embodiments, it will be understood by those skilled in the art that various changes may be made and equivalents may be substituted for elements thereof without departing from the scope of the disclosure. In addition, many modifications will be appreciated by those skilled in the art to adapt a particular instrument, situation or material to the teachings of the disclosure without departing from the essential scope thereof. Therefore, it is intended that the disclosure not be limited to the particular embodiment disclosed as the best mode contemplated for carrying out embodiments of this disclosure, but that the disclosed embodiments will include all embodiments falling within the scope of the appended claims.

What is claimed is:

1. A composition for densifying hair of a user, comprising:
a. silica in an amount between about 5.00 and 25.00 weight percent;
b. fiber in an amount between about 20.00 weight percent and 50.00 weight percent including a pigment deposited thereon and/or therein; and
c. mica in an amount between about 3.00 and 25.00 weight percent.

2. The composition of claim 1, further comprising titanium dioxide in an amount between about 0.10 and about 20.00 weight percent.

3. The composition of claim 1, further comprising iron oxide in an amount between about 0.10 and about 10.00 weight percent.

4. The composition of claim 1, wherein the fiber includes polyethylene terephthalate fiber.

5. The composition of claim 4, wherein the fiber has an average length between about 5 and 100 microns.

6. The composition of claim 4, wherein the fiber has an average length between about 10 and 30 microns.

7. The composition of claim 1, further comprising oat kernel protein in an amount between about 5.00 and 35.00 weight percent.

8. The composition of claim 7, wherein the oat kernel protein has an average particle size between about 50 and 100 microns.

9. The composition of claim 1, wherein the composition has a density between about 5.00 and about 8.00 grams per cubic inch.

10. The composition of claim 1, wherein particulate in the composition have particle sizes that span a range from about one micron to about 100 microns.

11. The composition of claim 1, further comprising dimethicone in an amount between about 0.1 and 1.0 weight percent having a viscosity between about 5 and 100 centistoke.

12. The composition of claim 1, further comprising at least one preservative in an amount between 0.5 and 1.0 weight percent.

13. The composition of claim 1, wherein the pigment causes a user's hair to appear light brown.

14. The composition of claim 1, wherein the pigment causes a user's hair to appear dark brown.

15. The composition of claim 1, wherein the pigment causes a user's hair to appear black.

16. The composition of claim 1, wherein the pigment causes a user's hair to appear gray.

17. The composition of claim 1, wherein the pigment causes a user's hair to appear black.

18. The composition of claim 1, wherein the pigment causes a user's hair to appear auburn.

19. A method of applying the composition of claim 1 to hair on a user's scalp to enhance the appearance of the density of the user's hair.

20. A treatment system including the composition of claim 1 in powder form in a pump actuated spray container.
